(19) United States
(12) Patent Application Publication
(10) Pub. No.: US 2003/0046008 A1
(43) Pub. Date: Mar. 6, 2003

(54) Method and apparatus for automated alignment of flexible structures

(75) Inventors: Haim Wolfson, Tel Aviv (IL); Maxim Shatsky, Tel Aviv (IL)

Correspondence Address: G.E. EHRLICH (1995) LTD., c/o ANTHONY CASTORINA, 2001 JEFFERSON DAVIS HIGHWAY, SUITE 207, ARLINGTON, VA, 22202, US

(73) Assignee: Ramot University Authority For Applied Research & Industrial Development Ltd.

(21) Appl. No.: 10/194,269

(22) Filed: Jul. 15, 2002

Related U.S. Application Data
(60) Provisional application No. 60/312,083, filed on Aug. 15, 2001.

(51) Int. Cl. G06G 7/48; G06G 7/58; G06F 19/00; G01N 33/48; G01N 33/50

(52) U.S. Cl. 702/19; 702/20; 703/11

(57) ABSTRACT

Apparatus for automated alignment of polymer structures, the polymer structures having predefined or non-predefined rigid portions and flexible portions. The apparatus comprises an input unit and a transforming unit, where the input unit receives a first polymer structure and a second polymer structure, and the transforming unit applies a semi-flexible transformation on at least a portion of the first polymer structure, at least a portion of the second polymer structure or at least portions of both the first and the second polymer structures. The transformation is done so as to at least partially superimpose the first polymer structure and the second polymer structure, hence to provide at least a partial alignment of the first and the second polymer structures.

### FIELD AND BACKGROUND OF THE INVENTION

The present invention relates to a method and apparatus for alignment of rigid subparts of flexible structures such as macromolecules and, more particularly but not exclusively, to a method and apparatus for efficient structural pattern detection of hinge regions and alignment of rigid subparts of macro and micro structures.

Informatics is the study and application of computer and statistical techniques for the management of information. In Genome projects, bioinformatics includes the development of methods to search databases fast and efficiently, to analyze nucleic acid sequence information, and to predict protein sequence and structure from DNA sequence data. Increasingly, molecular biology is shifting from the laboratory bench to the computer desktop. Advanced quantitative analyses, database comparisons, and computational algorithms are needed to explore the relationships between sequence, structure and phenotype. However a successful analysis has to better deal with the problem of protein structural alignment.

Proteins are linear polymers of amino acids. The polymerization reaction, which produces a protein, results in the loss of one molecule of water from each peptide bond formed (linking two adjacent amino acids), and hence proteins are often said to be composed of amino acid residues. Natural protein molecules may contain as many as 20 different types of amino acid residues, the sequence of which defines the so-called “primary sequence” of the protein. Proteins fold into a three-dimensional (3D) structure, which is determined both by the sequence of amino acids and by the protein's environment. Examination of the three-dimensional structure of numerous natural proteins has revealed a number of recurring patterns, the most common are known as alpha helices, parallel beta sheets and anti-parallel beta sheets, which define a second level of structural organization. The amino acids, the peptide bond and the above structures are further described in many biology text books, including, for example, in “Biochemistry”, third edition, L. Stryer, W. H. Freeman and Company, NY. Algorithms are available to predict these structures based on the primary sequence of a protein. However, these algorithms make correct predictions only in limited number of cases in which the number of available homology proteins is sufficiently large.

The biological properties of proteins are mainly affected by the proteins' three-dimensional configuration (structure), which determines the activity of enzymes, the capacity and specificity of binding proteins such as receptors and antibodies, and the structural attributes of receptor/ligand molecules. Hence, the protein structure stores significantly more information than its sequence, in particular during evolution where structures have been much better conserved than sequence (that is to say both in converging and diverging evolution). It would therefore be expected that protein structural alignment methods could supply significant information that cannot be received from sequence alignment methods.

Protein structures are determined using a variety of techniques including x-ray crystallography, neutron and electron diffractions, and nuclear magnetic resonance. In the past, the number of known protein structures was small and hence the need for efficient methods of structural alignment of proteins was minute, which need was accomplishable manually. The need for highly efficient structural alignment methods has become evident with the significant increase in the number of entries in protein structure databases, as well as with the progress of the structural Genomics efforts. Structure alignment methods also apply for computer assisted drug design in the process of structurally aligning ligands acting on a similar receptor.

In the prior art, the problem of protein structural alignment has been addressed by considering proteins to be aligned as rigid structures, typically through the exploitation of the amino acid sequence order. However, in general, proteins cannot be viewed as completely rigid structures, but rather as structures comprising rigid parts with flexible regions connecting therebetween.

There is thus a widely recognized need for, and it would be highly advantageous to have, a method for automated alignment of protein structures devoid of the above limitation and which takes into consideration the flexibility of protein structures.

### SUMMARY OF THE INVENTION

According to one aspect of the present invention there is provided an apparatus for automated alignment of polymer structures, the polymer structures having rigid portions and flexible portions, the apparatus comprising: (a) an input unit, for inputting a first polymer structure and a second polymer structure; and (b) a transforming unit for applying a semi-flexible transformation on at least a portion of the first polymer structure, at least a portion of the second polymer structure or at least portions of both the first and the second polymer structures, so as to at least partially superimpose the first polymer structure and the second polymer structure, hence to provide at least a partial alignment of the first and the second polymer structures.

According to further features in preferred embodiments of the invention described below, the transforming unit comprises a storage unit for holding a set of rigid transformations, one rigid transformation for each of the rigid portions.

According to another aspect of the present invention there is provided a method of automated alignment of polymer structures, the polymer structures having rigid portions and flexible portions, the method being executable by a computer and comprising: (a) obtaining a first polymer structure and a second polymer structure; and (b) applying a semi-flexible transformation on at least a portion of the first polymer structure, at least a portion of the second polymer structure or at least portions of both the first and the second polymer structures, so as to at least partially superimpose the first polymer structure and the second polymer structure, hence providing at least a partial alignment of the first and the second polymer structures.

According to further features in preferred embodiments of the invention described below, the applying semi-flexible transformation comprises using a set of rigid transformations, one rigid transformation for each of the rigid portions.

According to yet another aspect of the present invention there is provided a method of searching a database for structural homologues, the database including a plurality of protein structures, the method being executable by a computer and comprising: (a) inputting a query protein structure; (b) for each of the plurality of protein structures of the database, applying a semi-flexible transformation, so as to at least partially superimpose the query protein structure and each of the plurality of protein structures, hence providing at least a partial structural alignments of the query protein structure and each of the plurality of protein structures; and (c) issuing a result.

According to further features in preferred embodiments of the invention described below, the method further comprising, prior to step (c): (i) for each of the at least partial structural alignment, obtaining a score using a scoring function; and (ii) sorting the at least partial structural alignments with respect to the score, thereby providing an ordered set of at least partial structural alignments.

According to still further features in the described preferred embodiments step (c) comprises outputting at least a portion of the ordered set.

According to still further features in the described preferred embodiments the at least a portion of the ordered set comprises a list, the list comprising at least one protein structure of the database having the highest the score.

According to still further features in the described preferred embodiments the at least a portion of the ordered set comprises a list, the list comprising consecutive components of at least one protein structure of the database having the highest the score.

According to still further features in the described preferred embodiments the method further comprising defining rigid portions and flexible portions for each the protein structure of the database.

According to still further features in the described preferred embodiments the applying a semi-flexible transformation comprises using a set of rigid transformations, one rigid transformation for each the rigid portion of the protein structure of the database.

According to still another aspect of the present invention there is provided an apparatus for automated alignment of polymer structures, the apparatus comprising: (a) an input unit for receiving sequences of co-ordinates representative of three-dimensional structure of at least a first polymer structure and a second polymer structure, each represented by a sequence of co-ordinates; (b) a detector operable to select from each of the first and the second polymer structure at least one set of fragments, the detector being associated with transformation functionality to ensure that each fragment is transformable so that a fragment of the first polymer structure and a fragment of the second polymer structure are at least partially superimposed, thereby to detect at least one set of pairs of congruent fragments; (c) an associating unit for associating at least two of the pairs of congruent fragments, to form at least one set of associated pairs of fragments; and (d) a clustering unit for clustering each set of associated pairs of fragments to provide at least one congruent region represented by at least one associated pair of fragments; thereby providing at least a partial alignment of polymer structures.

According to further features in preferred embodiments of the invention described below, the detector further comprises: (iv) a query-length setter for setting a query-length; (v) a memory for holding instructions for setting the match list to define a first pair of substantially congruent fragments; and (vi) a storage unit for holding ones of the pairs of substantially congruent fragments.

According to an additional aspect of the present invention there is provided a method of automated alignment of polymer structures, the method being executable by a computer and comprising: (a) receiving a first polymer structure and a second polymer structure, each represented by a sequence of co-ordinates; (b) for each the polymer structure, detecting at least one set of fragments, wherein each fragment of the sequence is respectively transformable so that a fragment of the first polymer structure and a fragment of the second polymer structure are at least partially superimposed, thereby providing at least one set of pairs of substantially congruent fragments; (c) for each set of pairs of substantially congruent fragments, mutually associating at least two pairs of the set of pairs, thereby providing at least one set of associated pairs of fragments; and (d) for each set of pairs of substantially congruent fragments clustering each of the set of associated pairs of fragments, thereby providing at least one congruent region represented by at least one associated pair of fragments; hence, providing a partial alignment of polymer structures.

According to further features in preferred embodiments of the invention described below, the sequence of co-ordinates is ordered in accordance with an amino acid order of the protein.

According to still further features in the described preferred embodiments the first seed co-ordinate is a first co-ordinate of the first polymer.

According to still further features in the described preferred embodiments the second seed co-ordinate is a first co-ordinate of the second polymer.

According to yet an additional aspect of the present invention there is provided an apparatus for automated alignment of polymer structures, the apparatus comprising: (a) an input unit for receiving sequences of co-ordinates representative of three-dimensional structure of at least a first polymer structure and a second polymer structure, each represented by a sequence of co-ordinates; (b) a detector operable to select from each of the first and the second polymer structure at least one set of fragments, the detector being associated with transformation functionality to ensure that each fragment is transformable, using a rigid transformation, so that a fragment of the first polymer structure and a fragment of the second polymer structure are at least partially superimposed, thereby to detect at least one set of rigid transformations; (c) a transforming unit for applying at least one of the set of rigid transformations over a plurality of fragments of the first polymer structure, a plurality of fragments of the second polymer structure or a plurality of fragments of both the first and the second polymer structures, so as to at least partially superimpose the first polymer structure and the second polymer structure, thereby to provide at least a partial alignment of the first and the second polymer structures.

According to still further features in the described preferred embodiments the input unit comprises functionality to order the sequence of co-ordinates in accordance with an amino acid order of the first and the second protein structures.

According to still further features in the described preferred embodiments the transforming unit comprises: (i) a constructor, for constructing a bipartite graph having a plurality of vertices of a first kind and a plurality of vertices of a second kind, the constructor being operable to ensure that each vertex of the first kind represents a co-ordinate of the first polymer, and each vertex of the second kind represents a co-ordinate of the second polymer; (ii) a weighter, for obtaining a plurality of edges on the bipartite graph, each connecting one vertex of the first kind and one vertex of the second kind, thereby providing two connected vertices, thereby providing two connected co-ordinates.

According to still an additional aspect of the present invention there is provided a method of automated alignment of polymer structures, the method being executable by a computer and comprising: (a) receiving a first polymer structure and a second polymer structure, each represented by a sequence of co-ordinates; (b) for each the polymer structure, detecting at least one set of fragments, wherein each fragment of a respective sequence is respectively transformable using a rigid transformation, so that a fragment of the first polymer structure and a fragment of the second polymer structure are at least partially superimposed, thereby providing at least one set of pairs of substantially congruent fragments, each characterized by a rigid transformation, hence providing at least one rigid transformation; and (c) applying at least one of the at least one rigid transformation over a plurality of fragments of the first polymer structure, a plurality of fragments of the second polymer structure or a plurality of fragments of both the first and the second polymer structures, so as to at least partially superimpose the first polymer structure and the second polymer structure, thereby to provide at least a partial alignment of the first and the second polymer structures.

According to further features in preferred embodiments of the invention described below, each of the first polymer structure and the second polymer structure is independently a structure of a protein.

According to still further features in the described preferred embodiments the protein is selected from the group consisting of a ligand, a receptor, an enzyme and a structural protein.

According to a further aspect of the present invention there is provided a method of object recognition for computer vision, the object having a curve-like structure, the structure having rigid portions and flexible portions, the method being executable by a computer and comprising: (a) obtaining a first object structure and a second object structure; and (b) applying a semi-flexible transformation on at least a portion of the second object structure, so as to at least partially superimpose the first object and the second object, hence providing at least a partial recognition of the second object.

According to further features in preferred embodiments of the invention described below, the first and the second object structures are independently polymers.

According to still further features in the described preferred embodiments the applying semi-flexible transformation comprises using a set of rigid transformations, one for each of the rigid portions.

According to yet a further aspect of the present invention there is provided apparatus for computer recognition of objects by comparison, the object comprising a structure having rigid portions and flexible portions, the apparatus comprising: (a) an input unit, for inputting a first object structure and a second object structure; and (b) a transforming unit for applying a semi-flexible transformation on at least a portion of the second object structure, so as to at least partially superimpose the first object structure and the second object structure, hence to provide at least a partial recognition of the second object.

According to further features in preferred embodiments of the invention described below, the rigid portions are selected from the group consisting of predefined rigid portions and non-predefined rigid portions.

According to still further features in the described preferred embodiments the flexible portions are selected from the group consisting of predefined flexible portions and non-predefined flexible portions.

According to still further features in the described preferred embodiments the transforming unit comprises a storage unit for holding a set of rigid transformations, one for each of the rigid portions.

According to still a further aspect of the present invention there is provided an apparatus for object recognition for computer vision of objects having a curve-like structure, the apparatus comprising: (a) an input unit for receiving sequences of co-ordinates representative of three-dimensional structure of at least a first object structure and a second object structure, each represented by a sequence of co-ordinates; (b) a detector operable to select from each of the first and the second object structure at least one set of fragments, the detector being associated with transformation functionality to ensure that each fragment is transformable so that a fragment of the first object structure and a fragment of the second object structure are at least partially superimposed, thereby to detect at least one set of pairs of congruent fragments; (c) an associating unit for associating at least two of the pairs of congruent fragments, to form at least one set of associated pairs of fragments; and (d) a clustering unit for clustering each set of associated pairs of fragments to provide at least one congruent region represented by at least one associated pair of fragments; thereby providing at least a partial recognition of the second object.

According to further features in preferred embodiments of the invention described below, the storage unit is operable to hold two consecutive the pairs of substantially congruent fragments which are partially overlapped.

According to still further features in the described preferred embodiments the query-length setter comprises electronic-calculating functionality for setting the query-length equal to a subtraction of half of the overlap from a length of the match-list.

According to still further features in the described preferred embodiments the associating unit comprises: (i) a constructor, for constructing a graph having a plurality of vertices, each vertex representing one of a respective pair of congruent fragments; (ii) a weighter, for obtaining a plurality of directed edges on the graph each connecting two of the vertices thereby defining at each edge an incoming vertex and an outgoing vertex, and for weighting the edges using a scoring function, thereby providing a weighted acyclic directed graph; (iii) electronic-calculating functionality for applying a single-source shortest path algorithm to the weighted acyclic directed graph thereby to provide a plurality of paths; (iv) electronic-calculating functionality for classing the plurality of paths in accordance with a number of vertices on each of the plurality of paths, to define at least one class of paths, each class comprising at least one path; (v) electronic-calculating functionality for determining for each path of each class of paths, a value for path weight; and (vi) electronic-calculating functionality for sorting each class of paths using the values of path weight.

According to still further features in the described preferred embodiments the weighter comprises: (A) a selector, for selecting two of the plurality of vertices; (B) electronic-calculating functionality for determining whether corresponding pairs of substantially congruent fragments are in an ascending order, the ascending order being both with respect to the co-ordinates of the first object structure, and with respect to the co-ordinates of the second object structure; (C) an identifier, for determining a first gap between two consecutive fragments of the first object, and a second gap between two consecutive corresponding fragments of the second object; and (D) electronic-calculating functionality for comparing the first gap with a predetermined threshold MaxGap1 and for comparing the second gap with a predetermined threshold MaxGap2.

According to still further features in the described preferred embodiments: the constructor is operable to construct an additional virtual vertex; and the weighter is operable to obtain a virtual edge connecting the virtual vertex with all the plurality of vertices and to weight each the virtual edge using a virtual scoring function.

According to still further features in the described preferred embodiments the clustering unit comprises: (i) a storage unit for storing a query-region comprising at least one associated pair of fragments; (ii) a transforming unit for simultaneously transforming the query-region using a rigid transformation so as to obtain a superimposition of all of the associated pairs of fragments within the query-region; (iii) electronic-calculating functionality for determining a query-region root-mean-square deviation; (iv) a memory for holding instructions for setting one congruent region equal to the query-region; and (v) a storage unit for storing each congruent region.

According to still further features in the described preferred embodiments the instructions of part (iv) comprise: determining whether the query-region root-mean-square deviation is below a predetermined threshold MaxRMSD, and if so then setting one congruent region equal to the query-region.

According to still further features in the described preferred embodiments the clustering unit further comprising a query-region initiator for setting the query-region equal to a first associated pair of fragments.

According to still further features in the described preferred embodiments the clustering unit further comprising a query-region initiator for setting the query-region equal to an associated pair of fragments consecutive to an existing one of the congruent regions.

According to still a further aspect of the present invention there is provided a method of object recognition for computer vision, the object having a curve-like structure, the method being executable by a computer and comprising: (a) receiving a first object structure and a second object structure, each represented by a sequence of co-ordinates; (b) for each the object structure, detecting at least one set of fragments, wherein each fragment of a respective sequence is respectively transformable so that a fragment of the first object structure and a fragment of the second object structure are at least partially superimposed, thereby providing at least one set of pairs of substantially congruent fragments; (c) for each set of pairs of substantially congruent fragments, mutually associating at least two pairs of the set of pairs, thereby providing at least one set of associated pairs of fragments; and (d) for each set of pairs of substantially congruent fragments clustering each of the set of associated pairs of fragments, thereby providing at least one congruent region represented by at least one associated pair of fragments; hence providing at least a partial recognition of the second object.

According to further features in preferred embodiments of the invention described below, the at least partially superimposed comprises a small overall root-mean-square deviation.

According to still further features in the described preferred embodiments the overlap is smaller than a predetermined threshold MaxOverlap.

According to still further features in the described preferred embodiments the match-list is initiated by a seed comprising a first seed co-ordinate and a second seed co-ordinate.

According to still further features in the described preferred embodiments the first and the second seed co-ordinates are respectively consecutive to a previously defined pair of congruent fragments.

According to still further features in the described preferred embodiments the first seed co-ordinate is a first co-ordinate of the first object.

According to still further features in the described preferred embodiments the second seed co-ordinate is a first co-ordinate of the second object.

According to still further features in the described preferred embodiments the associating comprises: (i) constructing a graph having a plurality of vertices, each vertex representing one of the pair of congruent fragments; (ii) obtaining a plurality of directed edges on the graph each connecting two of the vertices and defining an incoming vertex and an outgoing vertex, wherein each the edge is weighted using a scoring function, thereby providing a weighted acyclic directed graph; (iii) applying a single-source shortest path algorithm to the weighted acyclic directed graph thereby providing a plurality of paths; (iv) classing the plurality of paths decreasingly in accordance with a number of vertices on each of the plurality of paths, thereby defining at least one class of paths, each class comprising at least one path; (v) for each class of paths, determining for the path, a value for path weight; and (vi) for each class of paths, sorting each path using the values of path weight; thereby providing at least one set of associated pairs of fragments.

According to still further features in the described preferred embodiments the obtaining a plurality of directed edges on the graph comprises: (A) selecting two of the vertices; (B) determining whether corresponding pairs of substantially congruent fragments are in an ascending order, the ascending order being both with respect to the co-ordinates of the first object structure, and with respect to the co-ordinates of the second object structure; (C) determining a first gap and a second gap; and (D) then, if the corresponding pairs of substantially congruent fragments are in the ascending order and if the first gap is smaller than a predetermined threshold MaxGap1 and if the second gap is smaller than a predetermined threshold MaxGap2, then obtaining a directed edge between the two vertices.

According to still further features in the described preferred embodiments each of the first and the second gap are respectively structurally dissimilar fragments of the first and the second object structures, the structurally dissimilar fragments being between the corresponding pairs of substantially congruent fragments which are in the ascending order.

According to still further features in the described preferred embodiments the scoring function is substantially: −(L+1−Δ)²+max(|Gap1|,|Gap2|)+∥Gap1|−|Gap2∥, wherein L is a length of the pair of substantially represented by the incoming vertex, where Δ is half of the overlap, where Gap1 is the first gap and where Gap2 is the second gap.

According to still further features in the described preferred embodiments the method further comprising adding a virtual vertex to the weighted acyclic directed graph, the virtual vertex being connected by a virtual edge to all of the vertices, wherein the virtual edge is weighted by a virtual scoring function.

According to still further features in the described preferred embodiments the virtual scoring function substantially equals zero.

According to still further features in the described preferred embodiments the clustering comprises: (i) establishing a query-region comprising a seed of an associated pair of fragments; (ii) concatenating an additional associated pair of fragments to the query-region; (iii) simultaneously transforming the query-region using a rigid transformation SO as to obtain a superimposition of all of the associated pairs of fragments within the query-region; (iv) determining a region root-mean-square deviation; and (v) if the region root-mean-square deviation is below a predetermined threshold MaxRMSD, then setting one congruent region equal to the query-region.

According to still further features in the described preferred embodiments steps (ii) to (v) are repeated at least once.

According to still further features in the described preferred embodiments the seed of associated pair of fragments of step (i) comprises a first associated pair of fragments.

According to still further features in the described preferred embodiments the seed of associated pair of fragments of step (i) comprises an associated pair of fragments consecutive to an existing one of the congruent regions.

According to still a further aspect of the present invention there is provided apparatus for object recognition for computer vision of objects having a curve-like structure, the apparatus comprising: (a) an input unit for receiving sequences of co-ordinates representative of three-dimensional structure of at least a first object structure and a second object structure, each represented by a sequence of co-ordinates; (b) a detector operable to select from each of the first and the second object structure at least one set of fragments, the detector being associated with transformation functionality to ensure that each fragment is transformable, using a rigid transformation, so that a fragment of the first object structure and a fragment of the second object structure are at least partially superimposed, thereby to detect at least one set of rigid transformations; (c) a transforming unit for applying at least one of the set of rigid transformations over a plurality of fragments of the first object structure, a plurality of fragments of the second object structure or a plurality of fragments of both the first and the second object structures, so as to at least partially superimpose the first object structure and the second object structure, thereby to provide at least a partial alignment of the first and the second object structures.

According to further features in preferred embodiments of the invention described below, the detector comprises: (i) a storage unit for holding a match-list comprising at least one element, each element comprising a pair of co-ordinates, one co-ordinate of the first object structure and one co-ordinate of the second object structure; (ii) electronic-calculating functionality for determining a root-mean-square deviation of a concatenated match-list comprising the match-list and at least one additional element; and (iii) a memory for holding instructions for setting the match-list equal to the concatenated match-list.

According to still further features in the described preferred embodiments the instructions comprise determining whether the root-mean-square deviation is below a predefined threshold MaxRMSD, and if so then setting the match-list equal to the concatenated match-list.

According to still further features in the described preferred embodiments the electronic-calculating functionality of the part (ii) is operable to select the at least one additional element from the group consisting of a consecutive element to the right of the match-list and a consecutive element to the left of the match-list.

According to still further features in the described preferred embodiments the detector further comprises: (iv) a query-length setter for setting a query-length; (v) a memory for holding instructions for setting the match list to define a first pair of congruent fragments; and (vi) a storage unit for holding the first pair of congruent fragments.

According to still further features in the described preferred embodiments the instructions comprise determining whether the query-length is above a predetermined threshold MinFragSize and if so then defining the first pair of substantially congruent fragments to be equal to the match-list.

According to still further features in the described preferred embodiments the query-length setter comprises electronic-calculating functionality for setting the query-length equal to a total number of elements of the match-list.

According to still further features in the described preferred embodiments the apparatus further comprising a match list initiator for setting the match list equal to a single element consecutive to a previously defined pair of congruent fragments.

According to still further features in the described preferred embodiments the transforming unit comprises: (i) a constructor, for constructing a bipartite graph having a plurality of vertices of a first kind and a plurality of vertices of a second kind, the constructor being operable to ensure that each vertex of the first kind represents one co-ordinate of the first object, and each vertex of the second kind represents one co-ordinate of the second object; (ii) a weighter, for obtaining a plurality of edges on the bipartite graph, each connecting one vertex of the first kind and one vertex of the second kind, thereby providing two connected vertices, thereby providing two connected co-ordinates.

According to still further features in the described preferred embodiments the weighter comprises: (A) a selector for selecting two non-connected vertices, one vertex of the first kind and one vertex of the second kind; (B) electronic-calculating functionality for determining a distance between the two non-connected vertices; (C) a memory for storing instructions for establishing an edge interconnecting the two non-connected vertices; and (D) a storage unit for holding the edge.

According to still further features in the described preferred embodiments the instructions of part (C) comprise determining whether the distance is below a predetermined threshold MaxDist, and if so then establishing the edge interconnecting the two non-connected vertices, thereby providing two connected vertices.

According to still further features in the described preferred embodiments the transforming unit further comprises electronic calculating functionality for finding a maximal number of vertex-disjoint edges, each vertex-disjoint edge is defined such that there is no common vertex between two vertex-disjoint edges.

According to still a further aspect of the present invention there is provided a method of object recognition for computer vision, the object having a curve-like structure, the method being executable by a computer and comprising: (a) receiving a first object structure and a second object structure, each represented by a sequence of co-ordinates; (b) for each the object structure, detecting at least one set of fragments, wherein each fragment of a respective sequence is respectively transformable using a rigid transformation, so that a fragment of the first object structure and a fragment of the second object structure are at least partially superimposed, thereby providing at least one set of pairs of substantially congruent fragments, each characterized by a rigid transformation, hence providing at least one rigid transformation; and (c) applying at least one of the at least one rigid transformation over a plurality of fragments of the first object structure, a plurality of fragments of the second object structure or a plurality of fragments of both the first and the second object structures, so as to at least partially superimpose the first object structure and the second object structure, thereby to provide at least a partial alignment of the first and the second object structures.

According to further features in preferred embodiments of the invention described below, steps (b) and (c) are repeated at least once.

According to still further features in the described preferred embodiments the at least partially superimposed sequence comprises a small overall root-mean-square deviation.

According to still further features in the described preferred embodiments step (b) comprises, (i) obtaining a match-list comprising at least one element, each element comprising a pair of co-ordinates, one co-ordinate of the first object structure and one co-ordinate of the second object structure; (ii) determining a root-mean-square deviation of a concatenated match-list comprising the match-list and at least one additional element; and (iii) if the root-mean-square deviation is below a predefined threshold MaxRMSD, then setting the match-list equal to the concatenated match-list.

According to still further features in the described preferred embodiments steps (ii) and (iii) are sequentially repeated at least once.

According to still further features in the described preferred embodiments the at least one additional element is selected from the group consisting of a consecutive element to the right of the match-list and a consecutive element to the left of the match-list.

According to still further features in the described preferred embodiments the method further comprising determining a query-length wherein if the query-length is above a predetermined threshold MinFragSize then setting the pair of substantially congruent fragments equal to the match-list.

According to still further features in the described preferred embodiments the query-length substantially equals a length of the match-list.

According to still further features in the described preferred embodiments the match-list comprises a single element consecutive to an existing one of the pairs of congruent fragments.

According to still further features in the described preferred embodiments step (c) comprises: (i) obtaining a bipartite graph having a plurality of vertices of a first kind and a plurality of vertices of a second kind, wherein each vertex of the first kind represents a co-ordinate of the first object, and each vertex of the second kind represents a co-ordinate of the second object; (ii) obtaining a plurality of edges on the bipartite graph, each connecting one vertex of the first kind and one vertex of the second kind, thereby providing two connected vertices, thereby providing two connected co-ordinates.

According to still further features in the described preferred embodiments step (ii) comprises respectively obtaining an edge interconnecting each vertex of the first kind representing a co-ordinate in the pair of substantially congruent fragments and each vertex of the second kind representing a co-ordinate in the pair of congruent fragments, thereby respectively connecting the rigid portion of the first object structure and the rigid portion of the second object structure.

According to still further features in the described preferred embodiments the method further comprising, for each two non-connected vertices, one of the vertices of the first kind and one of the vertices of the second kind: (A) determining a distance between the two non-connected vertices; and (B) if the distance is below a predetermined threshold MaxDist then establishing and edge interconnecting the two non-connected vertices, thereby providing two connected vertices.

According to still further features in the described preferred embodiments the method further comprising finding a maximal number of vertex-disjoint edges, each vertex-disjoint edge of the plurality vertex-disjoint edges being defined such that there is no common vertex between two the vertex-disjoint edges.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 shows a portion of a first polymer and a portion of a second polymer;

FIG. 2 is a schematic depiction of one embodiment of an apparatus for automated alignment of polymer structures according to the present invention;

FIG. 3 is a schematic depiction of another embodiment of an apparatus for automated alignment of polymer structures according to the present invention;

FIG. 4 is a structural similarity matrix, of which a diagonal fragment represents a pair of substantially congruent fragments according to the present invention;

FIG. 5 is a typical configuration of partial overlapping according to the present invention;

FIG. 6 is a flowchart, summarizing the steps of automated alignment of polymer structures according to the present invention;

FIG. 7 is a schematic depiction of yet another embodiment of an apparatus for automated alignment of polymer structures according to the present invention;

FIG. 8 is a computer image of a structural matching of a glutamine binding protein in an open (ligand-free) form and a histidine binding protein in complex form when it is bounded to histidine;

FIG. 9 is a secondary structure assignment of an alignment of a human Calmodulin and a Calmodulin in a complex form with a rabbit skeletal myosin light-chain kinase;

FIG. 10 is a secondary structure assignment of an alignment of an Adenylate kinase isoenzyme-3 and Adenylate kinase in a complex form with inhibitor AP=5=A; and

FIG. 11 is a computer image of a structural matching of an immunoglobulin Fab fragment and a murine T-cell antigen receptor.

### DESCRIPTION OF THE PREFERRED EMBODIMENTS

The present invention is of a method and apparatus for efficient structure alignment. Specifically, the present invention can be used for automated alignment of a query protein structure by searching a database of protein structures for structural homologues.

As used herein, the phrase “structural homologues” refers to homologues in three-dimensional conformation under semi flexible transformation and not necessarily to any homologous sequences.

The principles and operation of a method for automated alignment of polymer structures according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

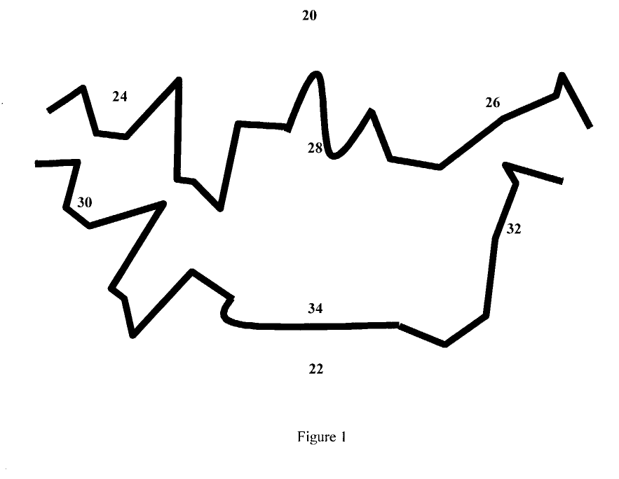

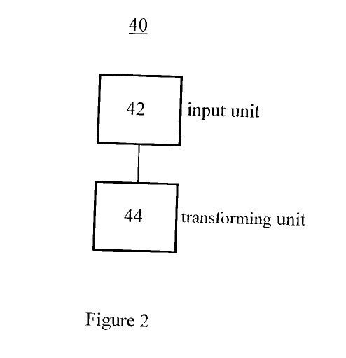

For purposes of better understanding the present invention, as illustrated in FIGS. 2-11 of the drawings, reference is first made to structural polymers as illustrated in FIG. 1.

FIG. 1 illustrates a portion of a first polymer 20 and a portion of a second polymer 22, which polymers are to be aligned using the present invention.

As used herein, the term “portion” refers to a sectional part of a polymer having a single rigid fragment or at least two rigid fragments interconnected by a flexible fragment or portion. The terms “fragment”, “segment” and “portion” are used herein interchangeably.

According to a preferred embodiment of the present invention, the structure of first polymer 20 and the structure of second polymer 22 are independently a structure of a protein, which can be, for example a ligand, a receptor, an enzyme or a structural protein.

The portion of first polymer 20 comprises a rigid fragment 24, a rigid fragment 26 and a flexible connection segment 28. The portion of second polymer 22 comprises a rigid fragment 30, a rigid fragment 32 and a flexible connection segment 34.

According to a preferred embodiment of the present invention there is provided an apparatus for automated alignment of polymer structures, which is referred to herein as apparatus 40.

As shown in FIG. 2, apparatus 40 includes an input unit 42, for inputting first polymer structure 20 and second polymer structure 22. Input unit 42 is connected to a transforming unit 44 which serves for applying a semi-flexible transformation as is further detailed hereinbelow.

As can be seen from FIG. 1, a rigid transformation, such as, but not limited to, a three-dimensional rotation and a three-dimensional translation, can be applied on rigid fragment 30 of second polymer 22 so as to at least partially superimpose rigid fragment 30 of second polymer 22 and rigid fragment 24 of first polymer 20. This transformation is presented in FIG. 1 as dotted paths connecting the two endpoints of rigid fragment 30 and rigid fragment 24.

Similarly, a rigid transformation, which is presented in FIG. 1 as dotted paths connecting the two endpoints of rigid fragment 32 and rigid fragment 26, can be applied on rigid fragment 32 of second polymer 22 so as to at least partially superimpose rigid fragment 34 of second polymer 22 and rigid fragment 26 of first polymer 20.

Unlike the rigid fragments, no rigid transformation would superimpose flexible connection segment 28 and flexible connection segment 34. Such an alignment of the indicated portion of first polymer 20 and the indicated portion of second polymer 22 is said to be “semi-flexible”. That is to say, the fragments themselves are transformed rigidly, however allowing flexibility within interconnecting flexible connection segments.

It should be understood that the phrase “semi-flexible” transformation also includes the degenerate case in which a each portion comprises a single rigid fragment, and a single rigid transformation is sufficient to superimpose the two portions.

Thus, according to a presently preferred embodiment of the invention, transforming unit 44 is designed and configured for applying a semi-flexible transformation on first polymer 20 and/or second polymer 22, so as to provide at least a partial alignment of first and second polymer structures.

Generally, a polymer may include a plurality of portions, which may increase the complexity of the alignment process, as the involved combinatorial factors grow rapidly with the length of the polymers or the numbers of alternating rigid and flexible portions thereof. It is to be understood that the most meaningful alignment would be of polymers comprising sufficiently large rigid fragments and sufficiently small flexible connection segments. In addition, it is preferred that the number of rigid fragments and/or flexible connections is minimized. A description of an efficient method and apparatus for alignment of polymers having a plurality of rigid fragments is herein provided.

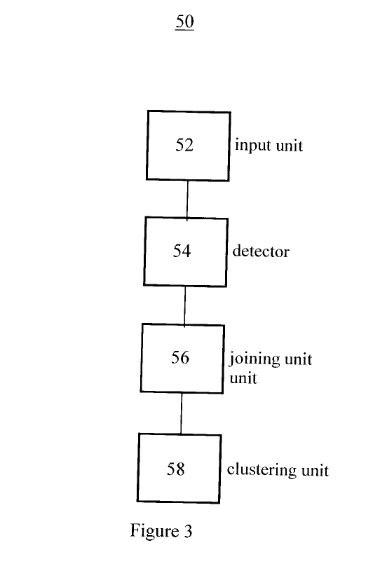

According to another preferred embodiment of the present invention there is provided an apparatus for automated alignment of polymer structures, which is referred to herein as apparatus 50, and reference is now made to FIG. 3, which is a simplified block diagram showing apparatus 50.

As shown in FIG. 3, apparatus 50 includes an input unit 52, for inputting a first polymer structure and a second polymer structure. Each of the polymer structures is represented by a sequence of co-ordinates over a system of co-ordinates, which can be for example, any three-dimensional system of co-ordinates. Input unit 52 is connected to a detector 54 which serves for detecting at least one appropriate set of transformable rigid fragments both for first polymer 20 and for second polymer 22. Each pair of rigid fragments such as, for example, rigid fragment 30 of second polymer 22 and rigid fragment 24 of first polymer 20 (FIG. 1), is considered as a pair of congruent fragments, hence at least one set of pairs of substantially congruent fragments is provided. The iteration procedure employed by detector 54 is further detailed hereinafter.

Detector 54 is connected to an associating unit 56 which serves for associating at least two pairs of congruent fragments, so as to provide a set of associated pairs of fragments. Specifically, the method further comprising obtaining a subset of pairs of congruent fragments, which corresponds to at least a partial alignment of first polymer 20 and second polymer 22. The at least partial alignment is the result of applying a set of rigid transformations, one on each of the pairs of substantially congruent fragments of the subset, allowing flexibility between two consecutive fragments, in a manner described hereinabove.

According to a preferred embodiment of the present invention all rigid transformations are applied on rigid fragments of one polymer, preferably second polymer 22, while fragments of the other polymer are not transformed. Alternatively, rigid transformations may also be applied to both fragments of first polymer 20 and second polymer 22.

As is further described hereinunder, due to the complexity of the process, there is more than one subset which may correspond to a sufficient alignment of the polymers. Hence, the output of associating unit 56, according to a preferred embodiment of the present invention, is at least one set of associated pairs of fragments, each corresponding to a set of rigid transformations on a sequence of rigid fragments interconnected by flexible fragments which cannot be matched. As stated, each of these rigid transformations may be different from all the other transformations, however some consecutive associated pairs of fragment may, up to a predefined degree of accuracy, share the same transformation.

Associating unit 56 is further connected to a clustering unit 58 for clustering all the consecutive associated pairs of fragments transformable under equal or close to equal rigid transformation, into one congruent region. Each congruent region corresponds to a different rigid transformation operable on all the associated pairs of fragments of the region. Hence, a set of congruent regions corresponds to a semi-flexible transformation of first polymer 20 or second polymer 22 or both first polymer 20 and second polymer 22.

A detailed description of the operations of apparatus 50, in accordance with a preferred embodiment of the present invention is herein provided.

Input unit 52 receives each polymer as a sequence of co-ordinates. Hence, each polymer can also be considered mathematically as an equal interval sequential sampling of a curve embedded in a three-dimensional space. A biological polymer is hereby interchangeably referred to as a mathematical curve, and an atom of a polymer is hereby interchangeably referred to as a point, which has been sampled from a mathematical curve.

As stated, detector 54 detects at least one appropriate set of transformable rigid fragments both for first polymer 20 and for second polymer 22. The detection is carried out using an iteration procedure, which comprises constructing a so-called “match-list”, where a starting element of the match-list is a pair of points, one of each polymer. At the beginning of the procedure the pair of points may be chosen arbitrarily, afterwards, when the iterative procedure evolves, other starting elements of the match-list may be selected, as further detailed hereinafter. Hence, the scope of the presently preferred embodiment of the invention is to extend the match-list to a maximal length, i.e., a maximal number of elements. Since the sample points are sequentially ordered, each point (except the endpoints of each curve) is positioned between a succeeding and a following point, one to the “right” and one to the “left”. At each step of the iteration procedure, the direction of extension of the match-list alternates, e.g., first to the “right” and then to the “left” or vice versa. An extension is performed by adding an element comprising a pair of consecutive points to existing points in the match-list in the desired direction. Each extension is followed by a rigid transformation, as described hereinabove, which is accompanied by an appropriate root-mean-square deviation calculation as further detailed hereinbelow. For each extension direction, the iteration procedure is continued while the obtained root-mean-square deviation is smaller than a predetermined threshold MaxRMSD, which is typically chosen to be between about 2 angstroms and about 4 angstroms. According to a preferred embodiment of the present invention the final match-list is considered to be a pair of substantially congruent fragments if and only if the size of the match-list is larger than a predetermined threshold MinFragSize.

Let {uₖ} and {vₖ}, k=1, . . . , n, be two sets of n points in three dimensional space, where the centroid of the set {uₖ} is at the origin. The root-mean-square deviation calculation is performed in accordance with the following formula:

$$\sqrt{\frac{1}{n} {\sum }_{k = 1}^{n} ( \vert {v}_{k} {\vert}^{2} + \vert {u}_{k} {\vert}^{2} ) - \frac{1}{{n}^{2}} \vert {\sum }_{k = 1}^{n} {v}_{k} {\vert}^{2} - \frac{2}{n} t r ( {( {A}^{T} A )}^{1 / 2} )}$$

where A is a 3×3 matrix defined as

$${A}_{i j} = {\sum }_{k = 1}^{n} {u}_{k}^{i} {v}_{k}^{j}$$

and where i and j are integer valued indices ranging from 1 to 3, denoting the i-th component and j-th component of the points uₖ and vₖ, respectively.

Once a pair of substantially congruent fragments is detected by detector 54, the above described iteration procedure is repeated, starting from a pair of points preferably consecutive to one of the endpoints of the previous match-list, until both endpoints of both polymer are reached. Thus, a single set of pairs of substantially congruent fragments is provided.

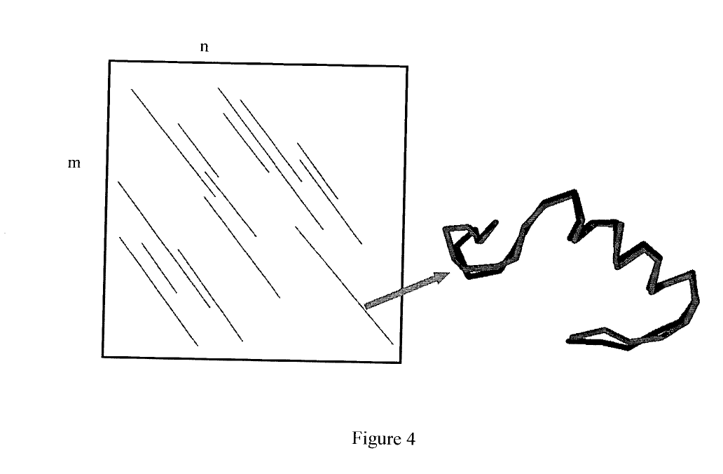

Detector 54 may detects more than one set, hence the above described procedure for detecting a single set is iteratively repeated, each time with a different starting element of the match-list. The operation of detector 54 is illustrated in FIG. 4 and may be better understood as follows. Considering a structural similarity n×m matrix M where n is the number of atoms in first polymer 20 and m is the number of atoms in second polymer 22, the detection process described hereinabove can be viewed as a motion along the diagonals of the matrix M. The initial starting atom pairs are elements on the diagonals of the matrix M.

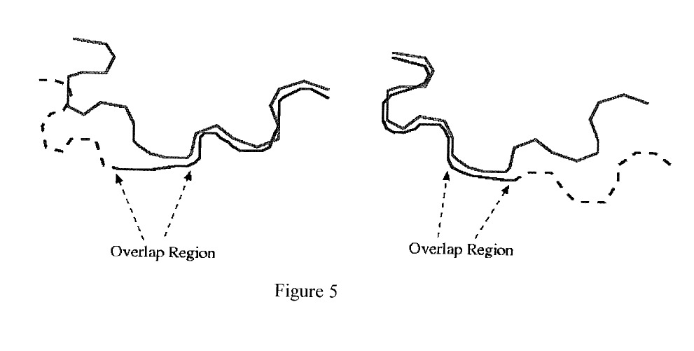

It is to be understood, that the extension procedure, employed by detector 54 for constructing the match-list may result in partial overlapping between two consecutive pairs of congruent fragments. Such overlapping, which may be characterized by an overlapping length, may be detected for example, when a rigid transformation produces a small torsion angle at a hinge point, permitting a small extension of rigid matching beyond the hinge point. A typical configuration of partial overlapping is demonstrated in FIG. 5.

In accordance with a preferred embodiment of the present invention, associating unit 56 further operates on the set of pairs of congruent fragments, to provide at least one associated pair of fragments. The set of all associated pairs of fragments is actually a subset of the set of pairs of congruent fragments, which subset is obtained by a number of optimization criteria and structural requirements. The structural requirements are listed herein, while the optimization criteria, manifested by the use of a scoring function, are described hereinafter. First, only ascending order pairs of substantially congruent fragments are associated, namely the ordering of the associated pairs of fragments is in accordance with the sequential ordering of the points on both curves. Second, two ascending order pairs of substantially congruent fragments may include a gap therebetween, which gap can be realized through a structurally dissimilar fragment between two ascending order pairs of substantially congruent fragments.

In addition to the ascending order requirement, according to a preferred embodiment of the present invention, a limited number of gaps in each polymer is allowed. This is ensured by introducing two predetermined thresholds MaxGap1 and MaxGap2, which are respectively the upper limits of the gaps of the first and second polymer, in terms of number of co-ordnates. A typical value for both MaxGap1 and MaxGap2 ranges between about 40 and about 60.

As stated, two consecutive pairs of substantially congruent fragments may include partial overlapping therebetween. According to a preferred embodiment of the present invention, only sufficiently small overlapping lengths are permitted within an associated pair of fragments. Specifically, each overlapping length between two consecutive pairs of substantially congruent fragments is required to be below a predetermined threshold referred to hereinbelow as MaxOverlap. A typical value for MaxOverlap is about 60% of the length of the overlapped pairs of congruent fragments. Preferably if the overlapped pairs have different lengths, MaxOverlap is related to the pair having the smaller length. The present embodiment also address the issue of “effective length”. Obviously, with a non zero overlapping length the effective length of two overlapped pairs of substantially congruent fragments is smaller that the number of elements which construct the pair. The effective length is preferably defined by equally correcting the total lengths (the number of elements) of the two overlapped pairs of congruent fragments. Specifically, denoting the total length of a pair of substantially congruent fragments by L and the overlapping length by 2Δ, then the effective length of a pair of substantially congruent fragments equals L-Δ. According to a presently preferred embodiment of the invention, the restrictions on the effective length are identical to the restrictions on L, i.e., the effective length is restricted to be below another threshold, referred to herein as MinFragSize. A typical value for MinFragSize ranges between about 5 and about 15.

Thus, the structural requirements, preset by associating unit 56, are the ascending order requirement, the limits on the gaps, the small overlapping length requirement and the sufficiently large effective length requirement. It is to be understood, that although in some applications of the present embodiment of the invention, the above structural requirements are applied simultaneously (i.e., using a Boolean “and” operation), each structural requirement of associating unit 56 may be also applied independently.

Once the structural requirement on each associated pair of fragments is established, the associating operation is performed. The associating operation is equivalent to a mathematical solution for the problem of obtaining short paths on an acyclic directed graph. Each pair of substantially congruent fragments is represented as a vertex on a graph, where at least two vertices of the graph are interconnected by a directed edge, which represents a flexible region connecting two consecutive pairs of congruent fragments. It is to be understood that in the special degenerate case described above, the acyclic graph includes a single vertex and zero number of edges.

Hence an acyclic directed graph is constructed. Since the edges of the acyclic directed graph have a unique direction, the two vertices connected by a single edge are identified as an incoming vertex (a “source”) and an outgoing vertex (a “target”). The direction of each edge of the acyclic directed graph is determined by the ascending order requirement, i.e., the direction of the interconnecting edge follows the sequential order of the points on both curves. The number of directed edges is controlled by the limits gaps, the small overlapping length requirement and the sufficiently large effective length requirement.

The present embodiment preferably detects at least one short path between two vertices over the acyclic directed graph. Preferably, while selecting a path along the acyclic directed graph, different weights may be assigned to each directed edge. That is to say that the length of a path is determined, both by the number of directed edges on the path and by the weight of each individual edge, where a small weight is considered to be a “reward” and a large weight is considered to be a “penalty”. The assigned weight w(e) for each directed edge is preferably in accordance with the following scoring function:

w(e)=−((L+1)−Δ)²+max(|Gap1|,|Gap2|)+∥Gap1|−|Gap2∥,

where L is the length of the pair of substantially congruent fragments which is preferably represented by the incoming vertex, and the parameters Gap1 and Gap2 represent the gaps in the first and second polymer, respectively. The above scoring function serves as a criterion for accepting or rejecting a particular pair of substantially congruent fragments to the optimal subset. As can be understood from the negative sign of the first term of the scoring function, large pairs of substantially congruent fragments are more likely to be accepted to the path. On the other hand large gaps increase the numerical value of the weight, hence reducing the likelihood of accepting a specific pair of substantially congruent fragments to the path.

Once weights are assigned to the directed edges, a weighted acyclic directed graph is constructed and paths over the weighted acyclic directed graph are scored in order to detect at least one short path. This may be accomplished for example by using an algorithm named “Single-Source Shortest Paths” which is known to be efficient, and may be found, for example in T. H. Cormen, C. E. Leiserson and R. L. Rivest, “Introduction to algorithms” (MIT Press 1990), chapter 25.4, the contents of which are hereby incorporated by reference. Alternatively, the at least one short path may be detected using an algorithm, named “All-Pairs Shortest Paths” which may be found in the above reference. In the preferred embodiment of the present invention the “Single-Source Shortest Paths” is used.

The “Single-Source Shortest Paths” algorithm is typically initiated from a single vertex, which is the source of all the constructed paths. The source may be any existing vertex on the weighted acyclic directed graph, however in a presently preferred embodiment of the invention, it is chosen to be an additional virtual vertex, which does not represent an existing pair of congruent fragments. The advantage of using an additional vertex is that no existing vertex is preferred a-priory. Thus, a virtual vertex is added and interconnected by virtual edges to all the existing vertices on the weighted acyclic directed graph. The assigned weight for each virtual edge is in accordance with a virtual scoring function, which may be any scoring function suitable for performing the “Single-Source Shortest Paths” algorithm. In a presently preferred embodiment of the invention, the virtual scoring function equals zero, i.e. all the virtual edges are assigned with a zero weight. Once the “Single-Source Shortest Paths” is applied using the virtual vertex, at least one path over the weighted acyclic directed graph is provided, which path automatically fulfills the predefined optimization criteria and structural requirements.

According to a preferred embodiment of the present invention, the paths are grouped according to the number of vertices in each path, hence at least one set of paths is obtained, each set is characterized by the number of vertices constructing the paths of the set. Each path is realized as an associated pair of fragments, hence at least one set of associated pairs of fragments is provided. Each set is preferably sorted in decreasing order, according to calculated as a sum of the weights of all the directed edges on the path.

In accordance with a preferred embodiment of the present invention, the apparatus further comprises clustering unit 58 for carrying out a clustering step. The clustering unit 58 is preferably employed on each associated pair of fragments of each set of associated pairs of fragments. The step of clustering comprises an iterative procedure of cluster construction, which is based on similarities in the applied rigid transformations. Let c be an existing cluster having at least one pair of substantially congruent fragments and let pi be the latest pair of substantially congruent fragments added to the cluster c. An additional pair of substantially congruent fragments pᵢ₊₁, consecutive to pᵢ, is now considered as to whether or not to be accepted in the cluster c. According to a preferred embodiment of the present invention, a rigid transformation is applied simultaneously on all the pairs of substantially congruent fragments in c as well as on pᵢ₊₁, so as to minimize the root-mean-square deviation. If the minimal root-mean-square deviation is below MaxRMSD then pᵢ₊₁ is accepted into the cluster, whereas if the minimal root-mean-square deviation is above MaxRMSD then pᵢ₊₁ is rejected from the cluster. In case of rejection, cluster c is defined as a congruent region, and the rejected pair of substantially congruent fragments initiates a singleton cluster on which the above iterative procedure is employed. According to a preferred embodiment of the present invention, for each associated pair of fragments, the step of clustering is initiated at the first pair of congruent fragments.

Hence, each associated pair of fragments comprises at least one congruent region characterized by a rigid transformation superimposing the congruent region of the first curve and the “twin” congruent region of the second curve with small root-mean-square deviation.

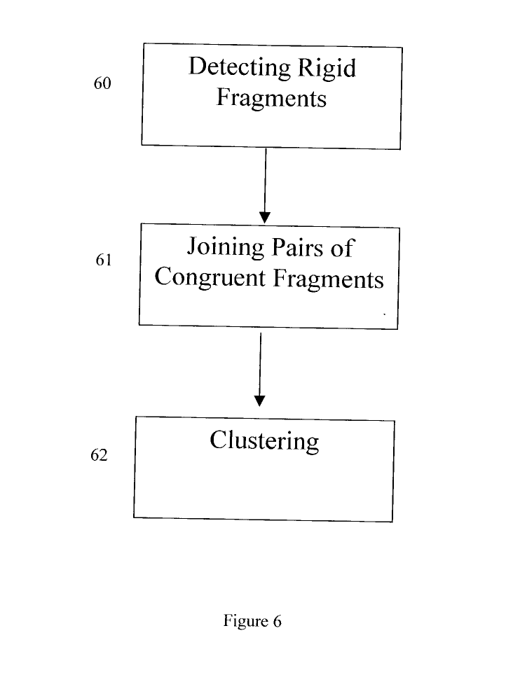

The basic steps of the presently preferred embodiment of the invention are hereby summarized with reference to FIG. 6 showing a flow chart of the operations. As shown in FIG. 6, block 60 represents the step of detecting at least one set of transformable rigid fragments both for the first curve and for the second curve. Block 60 is connected to block 61 which represents the step of associating at least two pairs of congruent fragments. Finally, block 61 is further connected to block 62 which represents a step of clustering all the consecutive associated pairs of fragments transformable under equal or close to equal rigid transformation, into one congruent region.

According to another preferred embodiment of the present invention, there is provided a method for object recognition for computer vision, which objects having a curve-like (one-dimensional) structure embedded in a three-dimensional space. It should be appreciated, that the principles and operation of a method for object recognition of computer vision are in one-to-one correspondence with the principles and operation of the alignment method of polymers described hereinabove. The input, according to a presently preferred embodiment of the invention, is a sequence of points forming a first curve and a sequence of points forming a second curve in a three-dimensional space. The curves may be realized, for example as a robot arm as obtained from a tracking system, a blood vessel as obtained from a medical imaging device or any other curve-like object which may be obtained from any visual system device, e.g. a camera.

In accordance with a presently preferred embodiment of the invention, the method comprises applying a semi-flexible transformation on at least a portion of the first curve, at least a portion of the second curve, or at least a portion of both the first curve and the second curve, as detailed hereinabove. Thus, the first curve and the second curve are at least partially superimposed, hence providing a recognition of the second curve with respect to the first curve.

According to another preferred embodiment of the present invention there is provided an apparatus for automated alignment of polymer structures, which is referred to herein as apparatus 64. The operations of apparatus 64 are partially incorporated with the operations of apparatus 50.

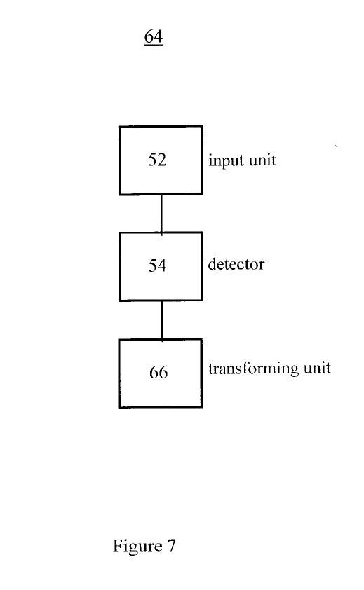

Reference is now made to FIG. 7, which is a simplified block diagram showing apparatus 64. Parts that are the same as those in previous figures are given the same reference numerals and are not described again except as necessary for an understanding of the present embodiment. As shown in FIG. 7, apparatus 64 includes input unit 52 and detector 54 the operation of which is described hereinabove for apparatus 50. Detector 54 is connected to a transforming unit 66, the operation of which is described hereinunder.

According to a presently preferred embodiment of the invention, detector 54 detects at least one set of transformable rigid fragments both for the first polymer and for the second polymer, through which a set of pairs of substantially congruent fragments is provided.

Each pair of substantially congruent fragments may be used, by transforming unit 66, as an initial alignment for a rigid matching of the two polymers. Specifically, transforming unit 66 stores the information of a rigid transformation applied on an individual pair of congruent fragments, and then uses that information to apply an identical or similar rigid transformation on the entire polymer. Such rigid transformation may provide at least partial superimposition of the first polymer and the second polymer. The superimposition may include the points on the pair of substantially congruent fragments and may also include other points not consecutive to the pair of congruent fragments. Hence, the set of pairs of substantially congruent fragments provides a set of candidate rigid transformations for transforming unit 66. Transforming unit 66 may use each of the candidate rigid transformations for superimposing the first polymer and the second polymer.

The structural comparison between the two rigid polymers, for each pair of congruent fragments, is equivalent to a mathematical solution for a problem of matching in a bipartite graph. A bipartite graph comprises a plurality of vertices of a first kind and a plurality of vertices of a second kind, and it may also include at least one edge interconnecting a vertex of the first kind and a vertex of the second kind. According to a preferred embodiment of the present invention, each point of the first curve is represented as a vertex of the first kind, and each point of the second curve is represented as a vertex of the second kind. Two unequal typed vertices are interconnected if the Euclidean distance between the points being represented by the two vertices is below a predetermined threshold MaxDist. Generally, the constructed graph may contain edges having common endpoints. According to a preferred embodiment of the present invention, the structural comparison between the two rigid polymers is accomplished by finding an optimal subset (e.g., of maximal size) of vertex-disjoint edges.

The solution to the mathematical problem of selecting a maximal number of such vertex-disjoint edges may be obtained, for example using an algorithm named “Maximal Cardinality Matching In The Bipartite Graph”, which can be found e.g. in Mehlhorn, “The LEDA Platform of Combinatorial and Geometric Computing”, (Cambridge University Press, 1999), the contents of which are hereby incorporated by reference.

In another embodiment of the present invention, yet another straightforward algorithm may be employed to efficiently select a maximal number of vertex-disjoint edges. In this preferred embodiment, an edge is added to the bipartite graph only if it does not create a common endpoint with another edge. The set of edges which have been assigned on the bipartite graph represents a match-list of sufficiently large size between points of the first curve and points of the second curve, thereby providing an alignment of the two curves.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Database and Computer Resources

The algorithm has been tested on samples from a database entitled “A database of macromolecular motions” to Gerstein, M. and Krebs, W., the contents of which are hereby incorporated by reference. The Gerstein's database has been published in Nucleic Acids Research 26 (18), and can also be found in the following web-site: http://bioinfo.mbb.yale.edu/MolMovDB/.

An additional database used is the SCOP database, the contents of which are hereby incorporated by reference. The SCOP database may be found in an article of Murzin, A., Brenner, S., Hubbard, T. and Chothia, C. (1995) titled “SCOP: a structural classification of proteins database for investigation of sequences and structures”, published in J. Mol. Biol. 247:536-540.

The experiments were conducted on a 400 MHz PENTIUM II processor, having an internal memory of 256 MB, and using a Linux operating system. Two additional computer programs were used for creating the accompanying figures: (a) Sayle, R. A. and Milner-White, E. J., “RasMol: bimolecular graphics for all”, published in Trends Biochem. Sci. 20(9):374; and (b) Humphrey, Dalke and Schulten “VMD viewer”, published in J. Mol. Graph 1996.

Predetermined Parameters

The values of the predetermined parameters used in the following examples are:

MaxMSD=3; MaxGap1=50; MaxGap2=50; and MinFragSize=10.

Summary of Experimental Results

The experimental results, which are further detailed in Examples 1-4, hereinunder, are summarized in Table 1. Table 1 includes 7 columns, in which the first column shows the PDB file names of matched molecules, the second column shows the size of each molecule, the third column shows the number of flexible regions found between the compared molecules, the fourth column shows the total number of matched C_{α} atoms, the fifth column shows the matched consecutive fragments, the sixth column shows the RMSD of the total matching set, and the seventh column shows the running time of the program. Groups of fragments in the fifth column which enclosed in square brackets represent clusters of fragments having the same 3-D transformation.

TABLE 1

| | Back- | No of | Match | | | |
|---|---|---|---|---|---|---|
| | bone | Flexible | List | | Total | |
| Protein Pair | Length | Regions | Size | Matched Rigid Fragments | RMSD | Time (sec) |
| 2bb (chain A) | 148 | 1 | 144 | (4 . . . 78)-(79 . . . 147) | 2.22 | 3.75 |
| 1c11 | 144 | | | (4 . . . 78)-(79 . . . 147) | | |
| 2bbm (chain A) | 148 | 3 | 147 | (2 . . . 25)-(26 . . . 63)-(64 . . . 76)-(77 . . . 148) | 2.43 | 4.48 |
| 1top | 162 | | | (12 . . . 35)-(36 . . . 73)-(76 . . . 88)-(90 . . . 161) | | |
| 2ak3 (chain A) | 226 | 2 | 205 | (6 . . . 120)-(121 . . . 164)-[(166 . . . 194)-(195 . . . 211)] | 2.53 | 6.9 |
| 1ake (chain A) | 214 | | | (1 . . . 115)-(118 . . . 161)-[(167 . . . 195)-(198 . . . 214)] | | |
| 2ak3 (chain A) | 226 | 1 | 184 | [(1 . . . 106)-(107-116)]-[(117 . . . 127)-(158 . . . 192)- | 2.31 | 5.09 |
| | | | | (193 . . . 214)] | | |
| 1uke | 193 | | | [(2 . . . 107)-(111-120)]-[(121 . . . 131)-(136 . . . 170)- | | |
| | | | | (172 . . . 193)] | | |
| 1bpd | 324 | 1 | 324 | (9 . . . 88)-(89 . . . 335) | 1.81 | 13.37 |
| 2bpg (chain A) | 324 | | | (9 . . . 88)-(89 . . . 335) | | |
| 1dpe | 507 | 2 | 507 | (1 . . . 262)-(263 . . . 480)-(481 . . . 507) | 0.58 | 25.89 |
| 1dpp (chain A) | 507 | | | (1 . . . 262)-(263 . . . 480)-(481 . . . 507) | | |
| 1ggg (chain A) | 220 | 2 | 220 | (5 . . . 87)-(88 . . . 180)-(181 . . . 224) | 0.96 | 7.25 |
| 1wdn (chain A) | 223 | | | (5 . . . 87)-(88 . . . 180)-(181 . . . 224) | | |
| 1ggg (chain A) | 220 | 2 | 220 | (5 . . . 89)-[(90 . . . 130)-(131 . . . 181)]-(182 . . . 224) | 2.07 | 7.46 |
| 1hpb | 239 | | | (7 . . . 91)-[(92 . . . 132)-(135 . . . 185)]-(192 . . . 234) | | |
| 1ncx | 162 | 3 | 161 | (1 . . . 35)-(36 . . . 68)-(69 . . . 92)-(93 . . . 161) | 2.7 | 4.93 |
| 1tnw (Model 1) | 162 | | | (1 . . . 35)-(36 . . . 68)-(69 . . . 92)-(93 . . . 161) | | |
| 1mcp (chain L) | 220 | 1 | 218 | (2 . . . 110)-(111 . . . 219) | 1.93 | 7.92 |
| 4fab (chain L) | 219 | | | (1 . . . 109)-(110 . . . 218) | | |
| 1mcp (chain L) | 220 | 1 | 213 | [(1 . . . 29)-(37 . . . 56)-(57 . . . 115)]-[(116 . . . 205)- | 2.4 | 9.5 |
| | | | | (206 . . . 220)] | | |
| 1tcr (chain B) | 236 | | | [(1 . . . 29)-(30 . . . 49)-(54 . . . 119)]-[(123 . . . 216)- | | |
| | | | | (232 . . . 246)] | | |
| 1lst | 239 | 2 | 238 | (1 . . . 90)-(91 . . . 177)-(178 . . . 238) | 1.35 | 8.30 |
| 2lao | 238 | | | (1 . . . 90)-(91 . . . 177)-(178 . . . 238) | | |
| 1lfh | 691 | 2 | 691 | (1 . . . 84)-(85 . . . 244)-(245 . . . 691) | 1.41 | 41.90 |
| 1lfg | 691 | | | (1 . . . 84)-(85 . . . 244)-(245 . . . 691) | | |
| 1ddt | 523 | 1 | 523 | (1 . . . 392)-(393 . . . 535) | 1.58 | 32.84 |
| 1mdt (chain A) | 523 | | | (1 . . . 392)-(393 . . . 535) | | |
| 3gap (chain A) | 208 | 1 | 205 | (1 . . . 130)-(131 . . . 205) | 1.8 | 6.79 |
| 3gap (chain B) | 205 | | | (1 . . . 130)-(131 . . . 205) | | |

### Example 1

Glutamine Binding Protein

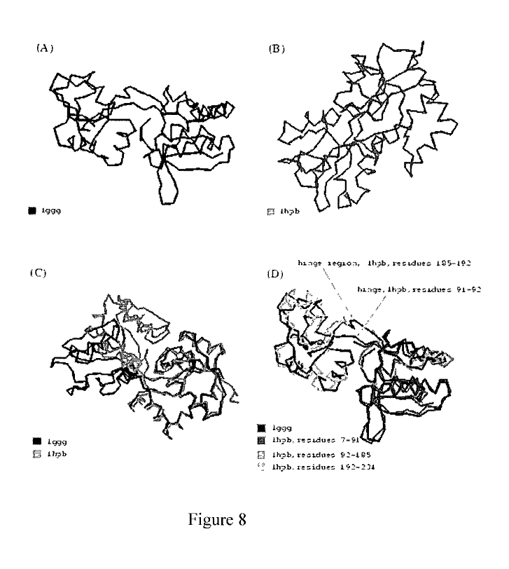

Two forms of proteins were taken from the Gerstein's database: (a) a glutamine binding protein in an open (ligant-free) form, which form is hereby denoted as “1ggg, chain A”; (b) a glutamine binding protein in a complex from when it is bounded to glutamine, which form is hereby denoted as “1wdn, chain A”. An additional protein was taken from the SCOP database: a histidine binding protein in complex from when it is bounded to histidine, which form is hereby denoted as “1hbp”. According the SCOP database, both the glutamine binding protein and the histidine binding protein belong to the family: “Phosphate binding protein-like”. The structures of “1ggg, chain A” and “1hbp” are shown in FIGS. 8(A) and 8(B), respectively.

First, “1ggg, chain A” was compared with “1wdn, chain A”. Two hinge conformations of one structure with respect to the other have been detected. The hinges are located at residues 87-88 and 180-181. The root-mean-square deviation of the total matching set is 0.96.

Second, “1ggg, chain A” was compared with “1hbp”, where four similar fragments were detected. Two fragments with similar transformations are separated by a turn located at residue 132-135 of “1hbp”, resulting in three matched clusters with total root-mean-square deviation of 2.07. A graphic illustration of the matching is shown in

FIGS. 8(C) and 8(D). FIG. 8(C) displays the best rigid superimposition of “1ggg, chain A” and “1hbp” and FIG. 8(D) displays a superimposition of the same samples, after a semi-flexible transformation. As can be seen in the figures, the unmatched region appearing on the left side of FIG. 8(C) is almost absent in FIG. 8(D), hence the alignment is almost complete.

### Example 2

Motion in Calmodulin

Calmodulin (CaM) is a C_{α}²⁺ binding protein, which is involved in a wide range of cellular C_{α}²⁺—dependent signaling pathways. Calmodulin is known to regulate the activity of large number of proteins including protein kinases, protein phosphatases, nitric oxide synthase, inositol triphosphate kinase, nicotinamide adenine dinucleotide kinase, cyclic nucleotide phosphodiesterase, C_{α}²⁺ pumps and protein involved in motility.

Two proteins were taken from the Gerstein's database: (a) a human Calmodulin which is hereby denoted as “1cll”; (b) a Calmodulin in a complex form with a rabbit skeletal myosin light-chain kinase hereby denoted as “2bbm chain A”. An additional protein was taken from the SCOP database: Troponin C protein which has a similar structure to Calmodulin, and is hereby denoted as “1top”.

First, “1cll” was compared with “2bbm chain A”, where a hinge motion in a α-helix was detected at a region of residues 78-79.

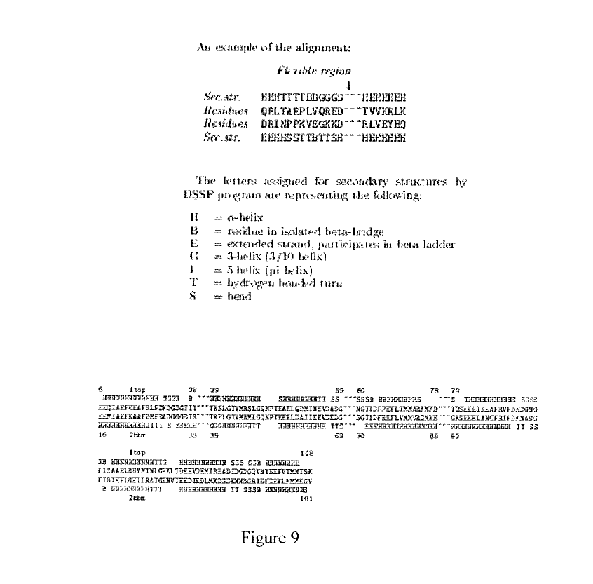

Secondly, “2bbm chain A” was compared with “1top”, where four similar rigid fragments separated by three hinge regions were detected. The alignment of “2bbm chain A” and “1top” is shown in FIG. 9.

### Example 3

Adenylate Kinase

Two forms of proteins were taken from the Gerstein's database: (a) Adenylate kinase isoenzyme-3 which is hereby denoted as “2ak3, chain A”; and (b) Adenylate kinase in a complex form with inhibitor AP=5=A which is hereby denoted as “1ake, chain A”. An additional protein was taken from the SCOP database: UMP/CMP kinase which is hereby denoted as “1uke”. Both Adenylate kinase and UMP/CMP are classified in the SCOP database as “Nucleotide and nucleoside kinases”.

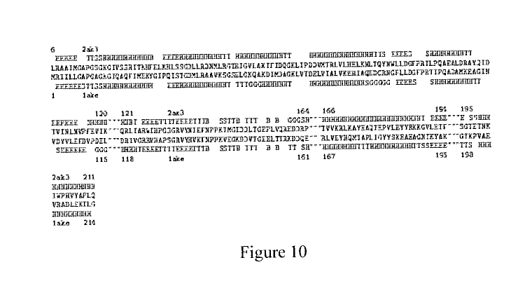

First, “2ak3, chain A” was compared with “1ake, chain A”, where four structurally similar regions were detected. The last two regions, having a similar transformation, were clustered into one group. The first flexible region is between residues 120 and 121 of “2ak3, chain A” and between residues 115 and 118 of “1ake, chain A”. The second flexible region is between residues 164 and 166 of “2ak3, chain A” and between residues 161 and 167 of “1ake, chain A”. A kinked helix at residues 164-188 was not detected, due to small overall conformational changes. The alignment of “2ak3, chain A” and “1ake, chain A” is shown in FIG. 10 (notation as in FIG. 9).

Second, “2ak3, chain A” was compared with “1uke”, where five structurally similar regions were detected. The first two regions were separated by a small loop but shared the same transformation hence clustered into one rigid matching. The remaining three regions also shared the same transformation and hence clustered into one matching set. The corresponding root-mean-square deviation for these clusters is 2.31. In the present example the value of the threshold Max RMSD was set to 2.5, as opposed to the value of 3, used in examples 1, 2 and 4 (below).

### Example 4

Immunoglobulin (Fab Elbow Joint)

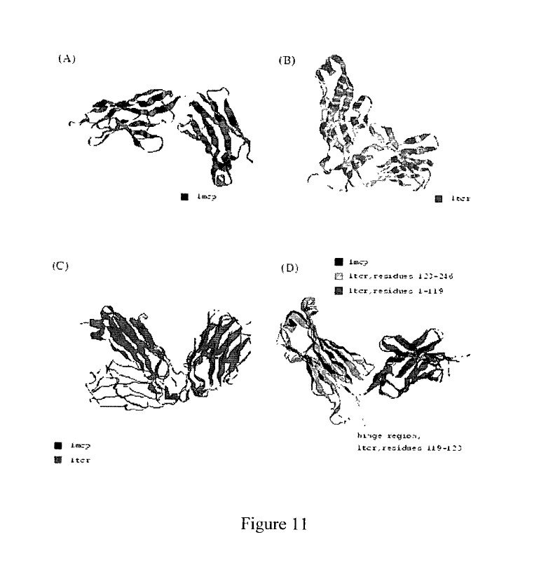

Two immunoglobulin Fab fragments were taken from the Gerstein's database: (a) a immunoglobulin Fab fragment which is hereby denoted as “1mcp, chain L” and (b) a immunoglobulin Fab fragment which is hereby denoted as “4fab, chain L”. Each chain of the above fragments is composed of two domains connected by an extended strand. In addition, a murine T-cell antigen receptor has been taken from the SCOP database, hereby denoted as “1tcr, chain B”. Accordingly the SCOP database, both “1mcp, chain L” and “1tcr, chain B” belong to a “V set domains (antibody variable domain-like)” family. A ribbon representation of the structures of “1mcp, chain L” and “1tcr, chain B” are shown in FIGS. 11(A) and 11(B), respectively.

First “1mcp, chain L” has been compared with “4fab, chain L”, where a flexible region was detected at residues 109-111. The corresponding root-mean-square deviation was 1.93.

Second “1mcp, chain L” was compared with “1tcr, chain B”, where two domains separated by a flexible part were detected. The corresponding root-mean-square deviation was 2.4.

A graphic illustration of the matching is shown in FIGS. 11(C) and 11(D). FIG. 11(C) displays the best rigid superimposition of “1mcp, chain L” and “1tcr, chain B”, and FIG. 8(D) displays a superimposition of the same fragments, after a semi-flexible transformation. As can be seen in the figures, the unmatched region appearing on the left side of FIG. 11(C) is almost absent in FIG. 11(D), hence “1tcr, chain B” is superimposed on “1mcp, chain L”.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. Apparatus for automated alignment of polymer structures, the polymer structures having rigid portions and flexible portions, the apparatus comprising:
(a) an input unit, for inputting a first polymer structure and a second polymer structure; and
(b) a transforming unit for applying a semi-flexible transformation on at least a portion of said first polymer structure, at least a portion of said second polymer structure or at least portions of both said first and said second polymer structures, so as to at least partially superimpose said first polymer structure and said second polymer structure, hence to provide at least a partial alignment of said first and said second polymer structures.

2. The apparatus of claim 1, wherein said rigid portions are selected from the group consisting of predefined rigid portions and non-predefined rigid portions.

3. The apparatus of claim 1, wherein said flexible portions are selected from the group consisting of predefined flexible portions and non-predefined flexible portions.

4. The apparatus of claim 1, wherein each of said first polymer structure and said second polymer structure is independently a structure of a protein.

5. The apparatus of claim 4, wherein said protein is selected from the group consisting of a ligand, a receptor, an enzyme and a structural protein.

6. The apparatus of claim 1, wherein said transforming unit comprises a storage unit for holding a set of rigid transformations, one rigid transformation for each of the rigid portions.

7. A method of automated alignment of polymer structures, the polymer structures having rigid portions and flexible portions, the method being executable by a computer and comprising:
(a) obtaining a first polymer structure and a second polymer structure; and
(b) applying a semi-flexible transformation on at least a portion of said first polymer structure, at least a portion of said second polymer structure or at least portions of both said first and said second polymer structures, so as to at least partially superimpose said first polymer structure and said second polymer structure, hence providing at least a partial alignment of said first and said second polymer structures.

8. The method of claim 7, wherein said rigid portions are selected from the group consisting of predefined rigid portions and non-predefined rigid portions.

9. The method of claim 7, wherein said flexible portions are selected from the group consisting of predefined flexible portions and non-predefined flexible portions.

10. The method of claim 7, wherein each of said first polymer structure and said second polymer structure is independently a structure of a protein.

11. The method of claim 10, wherein said protein is selected from the group consisting of a ligand, a receptor, an enzyme and a structural protein.

12. The method of claim 7, wherein said applying semi-flexible transformation comprises using a set of rigid transformations, one rigid transformation for each of the rigid portions.

13. A method of searching a database for structural homologues, the database including a plurality of protein structures, the method being executable by a computer and comprising:
(a) inputting a query protein structure;
(b) for each of said plurality of protein structures of said database, applying a semi-flexible transformation, so as to at least partially superimpose said query protein structure and each of said plurality of protein structures, hence providing at least a partial structural alignments of said query protein structure and each of said plurality of protein structures; and
(c) issuing a result.

14. The method of claim 13, further comprising, prior to step (c):
(i) for each of said at least partial structural alignment, obtaining a score using a scoring function; and
(ii) sorting said at least partial structural alignments with respect to said score, thereby providing an ordered set of at least partial structural alignments.

15. The method of claim 13, wherein said step (c) comprises outputting at least a portion of said ordered set.

16. The method of claim 15, wherein said at least a portion of said ordered set comprises a list, said list comprising at least one protein structure of the database having the highest said score.

17. The method of claim 14, wherein said at least a portion of said ordered set comprises a list, said list comprising consecutive components of at least one protein structure of the database having the highest said score.

18. The method of claim 13, further comprising defining rigid portions and flexible portions for each said protein structure of the database.

19. The method of claim 18, wherein said applying a semi-flexible transformation comprises using a set of rigid transformations, one rigid transformation for each said rigid portion of said protein structure of the database.

20. An apparatus for automated alignment of polymer structures, the apparatus comprising:
(a) an input unit for receiving sequences of co-ordinates representative of three-dimensional structure of at least a first polymer structure and a second polymer structure, each represented by a sequence of co-ordinates;
(b) a detector operable to select from each of said first and said second polymer structure at least one set of fragments, said detector being associated with transformation functionality to ensure that each fragment is transformable so that a fragment of said first polymer structure and a fragment of said second polymer structure are at least partially superimposed, thereby to detect at least one set of pairs of congruent fragments;
(c) an associating unit for associating at least two of said pairs of congruent fragments, to form at least one set of associated pairs of fragments; and
(d) a clustering unit for clustering each set of associated pairs of fragments to provide at least one congruent region represented by at least one associated pair of fragments;
thereby providing at least a partial alignment of polymer structures.

21. The apparatus of claim 20, wherein said polymer structures are protein structures.

22. The apparatus of claim 21, wherein said input unit comprises functionality to order said sequence of co-ordinates in accordance with an amino acid order of said first and said second protein structures.

23. The apparatus of claim 20, wherein said detector comprises:
(i) a storage unit for holding a match-list comprising at least one element, each element comprising a pair of co-ordinates, respectively being one co-ordinate of said first polymer structure and one co-ordinate of said second polymer structure;
(ii) electronic-calculating functionality for determining a root-mean-square deviation of a concatenated match-list comprising said match-list and at least one additional element; and
(iii) a memory for holding instructions for setting said match-list equal to said concatenated match-list.

24. The apparatus of claim 23, wherein said instructions comprise determining whether said root-mean-square deviation is below a predefined threshold MaxRMSD, and if so then setting said match-list equal to said concatenated match-list.

25. The apparatus of claim 23, wherein said electronic-calculating functionality of said part (ii) is operable to select said at least one additional element from the group consisting of a consecutive element to the right of said match-list and a consecutive element to the left of said match-list.

26. The apparatus of claim 23, wherein said detector further comprises:
(iv) a query-length setter for setting a query-length;
(v) a memory for holding instructions for setting said match list to define a first pair of substantially congruent fragments; and
(vi) a storage unit for holding ones of said pairs of substantially congruent fragments.

27. The apparatus of claim 26, wherein said instructions comprise determining whether said query-length is above a predetermined threshold MinFragSize and if so then defining said first pair of substantially congruent fragments to be equal to said match-list.

28. The apparatus of claim 26, wherein said query-length setter comprises electronic-calculating functionality for setting said query-length equal to a total number of elements of said match-list.

29. The apparatus of claim 26, wherein said storage unit is operable to hold two consecutive pairs of substantially congruent fragments which are partially overlapped.

30. The apparatus of claim 29, wherein said overlap is smaller than a predetermined threshold MaxOverlap.

31. The apparatus of claim 29, wherein said query-length setter comprises electronic-calculating functionality for setting said query-length equal to a subtraction of half of said overlap from a length of said match-list.

32. The apparatus of claim 26, further comprising a match list initiator for setting said match list equal to a single element consecutive to a previously defined pair of substantially congruent fragments.

33. The apparatus of claim 20, wherein said associating unit comprises:
(i) a constructor, for constructing a graph having a plurality of vertices, each vertex representing one of a respective pair of substantially congruent fragments;
(ii) a weighter, for obtaining a plurality of directed edges on said graph each connecting two of said vertices thereby defining at each edge an incoming vertex and an outgoing vertex, and for weighting said edges using a scoring function, thereby providing a weighted acyclic directed graph;
(iii) electronic-calculating functionality for applying a single-source shortest path algorithm to said weighted acyclic directed graph thereby to provide a plurality of paths;
(iv) electronic-calculating functionality for classing said plurality of paths in accordance with a number of vertices on each of said plurality of paths, to define at least one class of paths, each class comprising at least one path;
(v) electronic-calculating functionality for determining for each path of each class of paths, a value for path weight; and
(vi) electronic-calculating functionality for sorting each class of paths using said values of path weight.

34. The apparatus of claim 33, wherein said weighter comprises:
(A) a selector, for selecting two of said plurality of vertices;
(B) electronic-calculating functionality for determining whether corresponding pairs of substantially congruent fragments are in an ascending order, said ascending order being both with respect to said co-ordinates of said first polymer structure, and with respect to said co-ordinates of said second polymer structure;
(C) an identifier, for determining a first gap between two consecutive fragments of said first polymer, and a second gap between two consecutive corresponding fragments of said second polymer; and
(D) electronic-calculating functionality for comparing said first gap with a predetermined threshold MaxGap1 and for comparing said second gap with a predetermined threshold MaxGap2.

35. The apparatus of claim 34, wherein said storage unit is operable to hold two consecutive pairs of substantially congruent fragments which are partially overlapped.

36. The apparatus of claim 35, wherein said scoring function is substantially:
−(L+1−Δ)²+max(|Gap1|,|Gap2|)+∥Gap1|−|Gap2∥,
wherein:
L is a length of said pair of substantially congruent fragments, represented by said incoming vertex,
Δ is half of said overlap,
Gap1 is said first gap, and
Gap2 is said second gap.

37. The apparatus of claim 33, wherein:
said constructor is operable to construct an additional virtual vertex; and
said weighter is operable to obtain a virtual edge connecting said virtual vertex with all said plurality of vertices and to weight each said virtual edge using a virtual scoring function.

38. The apparatus of claim 37, wherein said virtual scoring function substantially equals zero.

39. The apparatus of claim 20, wherein said clustering unit comprises:
(i) a storage unit for storing a query-region comprising at least one associated pair of fragments;
(ii) a transforming unit for simultaneously transforming said query-region using a rigid transformation so as to obtain a superimposition of all of said associated pairs of fragments within said query-region;
(iii) electronic-calculating functionality for determining a query-region root-mean-square deviation;
(iv) a memory for holding instructions for setting one congruent region equal to said query-region; and
(v) a storage unit for storing each congruent region.

40. The apparatus of claim 39, wherein said instructions of part (iv) comprise: determining whether said query-region root-mean-square deviation is below a predetermined threshold MaxRMSD, and if so then setting one congruent region equal to said query-region.

41. The apparatus of claim 39, wherein said clustering unit further comprising a query-region initiator for setting said query-region equal to a first associated pair of fragments.

42. The apparatus of claim 39, wherein said clustering unit further comprising a query-region initiator for setting said query-region equal to an associated pair of fragments consecutive to an existing one of said congruent regions.

43. A method of automated alignment of polymer structures, the method being executable by a computer and comprising:
(a) receiving a first polymer structure and a second polymer structure, each represented by a sequence of co-ordinates;
(b) for each said polymer structure, detecting at least one set of fragments, wherein each fragment of said sequence is respectively transformable so that a fragment of said first polymer structure and a fragment of said second polymer structure are at least partially superimposed, thereby providing at least one set of pairs of substantially congruent fragments;
(c) for each set of pairs of substantially congruent fragments, mutually associating at least two pairs of said set of pairs, thereby providing at least one set of associated pairs of fragments; and
(d) for each set of pairs of substantially congruent fragments clustering each of said set of associated pairs of fragments, thereby providing at least one congruent region represented by at least one associated pair of fragments;
hence, providing a partial alignment of polymer structures.

44. The method of claim 43, wherein each of said first polymer structure and said second polymer structure is independently a structure of a protein.

45. The method of claim 44, wherein said protein is selected from the group consisting of a ligand, a receptor, an enzyme and a structural protein.

46. The method of claim 44, wherein said sequence of co-ordinates is ordered in accordance with an amino acid order of said protein.

47. The method of claim 43, wherein said at least partially superimposed sequence comprises a small overall root-mean-square deviation.

48. The method of claim 43, wherein step (b) comprises:
(i) obtaining a match-list comprising at least one element, each element comprising a pair of co-ordinates, one co-ordinate of said first polymer structure and one co-ordinate of said second polymer structure;
(ii) determining a root-mean-square deviation of a concatenated match-list comprising said match-list and at least one additional element; and
(iii) if said root-mean-square deviation is below a predefined threshold MaxRMSD, then setting said match-list equal to said concatenated match-list.

49. The method of claim 48, wherein steps (i)-(iii) are sequentially repeated at least once.

50. The method of claim 48, wherein said at least one additional element is selected from the group consisting of a consecutive element to the right of said match-list and a consecutive element to the left of said match-list.

51. The method of claim 49, further comprising determining a query-length wherein if said query-length is above a predetermined threshold MinFragSize then defining said pair of substantially congruent fragments to be equal to said match-list.

52. The method of claim 49, wherein said query-length substantially equals a length of said match-list.

53. The method of claim 51, wherein two consecutive said pairs of substantially congruent fragments are partially overlapped.

54. The method of claim 53, wherein said overlap is smaller than a predetermined threshold MaxOverlap.

55. The method of claim 53, wherein said query-length equals the subtraction of half of said overlap from a length of said match-list.

56. The method of claim 51, wherein said match-list is initiated by a seed comprising a first seed co-ordinate and a second seed co-ordinate.

57. The method of claim 56, wherein said first and said second seed co-ordinates are respectively consecutive to a previously defined pair of congruent fragments.

58. The method of claim 56, wherein said first seed co-ordinate is a first co-ordinate of said first polymer.

59. The method of claim 56, wherein said second seed co-ordinate is a first co-ordinate of said second polymer.

60. The method of claim 43, wherein said associating comprises:
(i) constructing a graph having a plurality of vertices, each vertex representing one of said pair of congruent fragments;
(ii) obtaining a plurality of directed edges on said graph each connecting two of said vertices and defining an incoming vertex and an outgoing vertex, wherein each said edge is weighted using a scoring function, thereby providing a weighted acyclic directed graph;
(iii) applying a single-source shortest path algorithm to said weighted acyclic directed graph thereby providing a plurality of paths;
(iv) classing said plurality of paths decreasingly in accordance with a number of vertices on each of said plurality of paths, thereby defining at least one class of paths, each class comprising at least one path;
(v) for each class of paths, determining for each path, a value for path weight; and
(vi) for each class of paths, sorting each path using said values of path weight;
thereby providing at least one set of associated pairs of fragments.

61. The method of claim 60, wherein said obtaining a plurality of directed edges on said graph comprises:
(A) selecting two of said vertices;
(B) determining whether corresponding pairs of substantially congruent fragments are in an ascending order, said ascending order being both with respect to said co-ordinates of said first polymer structure, and with respect to said co-ordinates of said second polymer structure;
(C) determining a first gap and a second gap; and
(D) then, if said corresponding pairs of substantially congruent fragments are in said ascending order and if said first gap is smaller than a predetermined threshold MaxGap1 and if said second gap is smaller than a predetermined threshold MaxGap2, then obtaining a directed edge between said two vertices.

62. The method of claim 61, wherein each of said first and said second gap are respectively structurally dissimilar fragments of said first and said second polymer structures, said structurally dissimilar fragments being between said corresponding pairs of substantially congruent fragments which are in said ascending order.

63. The method of claim 61, wherein two consecutive pairs of substantially congruent fragments are partially overlapped.

64. The method of claim 63, wherein said scoring function is substantially:
−(L+1−Δ)²+max(|Gap1|,|Gap2|)+∥Gap1|−|Gap2∥,
wherein L is a length of said pair of substantially congruent fragments represented by said incoming vertex, where Δ is half of said overlap, where Gap1 is said first gap and where Gap2 is said second gap.

65. The method of claim 60, further comprising adding a virtual vertex to said weighted acyclic directed graph, said virtual vertex being connected by a virtual edge to all of said vertices, wherein said virtual edge is weighted by a virtual scoring function.

66. The method of claim 65, wherein said virtual scoring function substantially equals zero.

67. The method of claim 43, wherein said clustering comprises:
(i) establishing a query-region comprising a seed of an associated pair of fragments;
(ii) concatenating an additional associated pair of fragments to said query-region;
(iii) simultaneously transforming said query-region using a rigid transformation so as to obtain a superimposition of all of said associated pairs of fragments within said query-region;
(iv) determining a region root-mean-square deviation; and
(v) if said region root-mean-square deviation is below a predetermined threshold MaxRMSD, then setting one congruent region equal to said query-region.

68. The method of claim 67, wherein steps (ii) to (v) are repeated at least once.

69. The method of claim 67, wherein said seed of associated pair of fragments of step (i) comprises a first associated pair of fragments.

70. The method of claim 67, wherein said seed of associated pair of fragments of step (i) comprises an associated pair of fragments consecutive to an existing one of said congruent regions.

71. Apparatus for automated alignment of polymer structures, the apparatus comprising:
(a) an input unit for receiving sequences of co-ordinates representative of three-dimensional structure of at least a first polymer structure and a second polymer structure, each represented by a sequence of co-ordinates;
(b) a detector operable to select from each of said first and said second polymer structure at least one set of fragments, said detector being associated with transformation functionality to ensure that each fragment is transformable, using a rigid transformation, so that a fragment of said first polymer structure and a fragment of said second polymer structure are at least partially superimposed, thereby to detect at least one set of rigid transformations;
(c) a transforming unit for applying at least one of said set of rigid transformations over a plurality of fragments of said first polymer structure, a plurality of fragments of said second polymer structure or a plurality of fragments of both said first and said second polymer structures, so as to at least partially superimpose said first polymer structure and said second polymer structure, thereby to provide at least a partial alignment of said first and said second polymer structures.

72. The apparatus of claim 71, wherein said polymer structures are protein structures.

73. The apparatus of claim 72, wherein said input unit comprises functionality to order said sequence of co-ordinates in accordance with an amino acid order of said first and said second protein structures.

74. The apparatus of claim 71, wherein said detector comprises:
(i) a storage unit for holding a match-list comprising at least one element, each element comprising a pair of co-ordinates, one co-ordinate of said first polymer structure and one co-ordinate of said second polymer structure;
(ii) electronic-calculating functionality for determining a root-mean-square deviation of a concatenated match-list comprising said match-list and at least one additional element; and
(iii) a memory for holding instructions for setting said match-list equal to said concatenated match-list.

75. The apparatus of claim 74, wherein said instructions comprise determining whether said root-mean-square deviation is below a predefined threshold MaxRMSD, and if so then setting said match-list equal to said concatenated match-list.

76. The apparatus of claim 74, wherein said electronic-calculating functionality of said part (ii) is operable to select said at least one additional element from the group consisting of a consecutive element to the right of said match-list and a consecutive element to the left of said match-list.

77. The apparatus of claim 74, wherein said detector further comprises:
(iv) a query-length setter for setting a query-length;
(v) a memory for holding instructions for setting said match list to define a first pair of congruent fragments; and
(vi) a storage unit for holding said first pair of congruent fragments.

78. The apparatus of claim 77, wherein said instructions comprise determining whether said query-length is above a predetermined threshold MinFragSize and if so then defining said first pair of substantially congruent fragments to be equal to said match-list.

79. The apparatus of claim 77, wherein said query-length setter comprises electronic-calculating functionality for setting said query-length equal to a total number of elements of said match-list.

80. The apparatus of claim 77, further comprising a match list initiator for setting said match list equal to a single element consecutive to a previously defined pair of congruent fragments.

81. The apparatus of claim 71, wherein said transforming unit comprises:
(i) a constructor, for constructing a bipartite graph having a plurality of vertices of a first kind and a plurality of vertices of a second kind, said constructor being operable to ensure that each vertex of said first kind represents a co-ordinate of said first polymer, and each vertex of said second kind represents a co-ordinate of said second polymer;
(ii) a weighter, for obtaining a plurality of edges on said bipartite graph, each connecting one vertex of said first kind and one vertex of said second kind, thereby providing two connected vertices, thereby providing two connected co-ordinates.

82. The apparatus of claim 81, wherein said weighter comprises:
(A) a selector for selecting two non-connected vertices, one vertex of said first kind and one vertex of said second kind;
(B) electronic-calculating functionality for determining a distance between said two non-connected vertices;
(C) a memory for storing instructions for establishing an edge interconnecting said two non-connected vertices; and
(D) a storage unit for holding said edge.

83. The apparatus of claim 82, wherein said instructions of part (C) comprise determining whether said distance is below a predetermined threshold MaxDist, and if so then establishing said edge interconnecting said two non-connected vertices, thereby providing two connected vertices.

84. The apparatus of claim 82, wherein said transforming unit further comprises electronic calculating functionality for finding a maximal number of vertex-disjoint edges, each vertex-disjoint edge of said plurality vertex-disjoint edges being defined such that there is no common vertex between two said vertex-disjoint edges.

85. A method of automated alignment of polymer structures, the method being executable by a computer and comprising:
(a) receiving a first polymer structure and a second polymer structure, each represented by a sequence of co-ordinates;
(b) for each said polymer structure, detecting at least one set of fragments, wherein each fragment of a respective sequence is respectively transformable using a rigid transformation, so that a fragment of said first polymer structure and a fragment of said second polymer structure are at least partially superimposed, thereby providing at least one set of pairs of substantially congruent fragments, each characterized by a rigid transformation, hence providing at least one rigid transformation; and
(c) applying at least one of said at least one rigid transformation over a plurality of fragments of said first polymer structure, a plurality of fragments of said second polymer structure or a plurality of fragments of both said first and said second polymer structures, so as to at least partially superimpose said first polymer structure and said second polymer structure, thereby to provide at least a partial alignment of said first and said second polymer structures.

86. The method of claim 85, wherein steps (b) and (c) are repeated at least once.

87. The method of claim 85, wherein each of said first polymer structure and said second polymer structure is independently a structure of a protein.

88. The method of claim 87, wherein said protein is selected from the group consisting of a ligand, a receptor, an enzyme and a structural protein.

89. The method of claim 85, wherein said at least partially superimposed sequence comprises a small overall root-mean-square deviation.

90. The method of claim 85, wherein step (b) comprises,
(i) obtaining a match-list comprising at least one element, each element comprising a pair of co-ordinates, one co-ordinate of said first polymer structure and one co-ordinate of said second polymer structure;
(ii) determining a root-mean-square deviation of a concatenated match-list comprising said match-list and at least one additional element; and
(iii) if said root-mean-square deviation is below a predefined threshold MaxRMSD, then setting said match-list equal to said concatenated match-list.

91. The method of claim 90, wherein steps (ii) and (iii) are sequentially repeated at least once.

92. The method of claim 90, wherein said at least one additional element is selected from the group consisting of a consecutive element to the right of said match-list and a consecutive element to the left of said match-list.

93. The method of claim 91, further comprising determining a query-length wherein if said query-length is above a predetermined threshold MinFragSize then setting said pair of substantially congruent fragments equal to said match-list.

94. The method of claim 93, wherein said query-length substantially equals a length of said match-list.

95. The method of claim 90, wherein said match-list comprises a single element consecutive to an existing one of said pairs of congruent fragments.

96. The method of claim 85, wherein step (c) comprises:
(i) obtaining a bipartite graph having a plurality of vertices of a first kind and a plurality of vertices of a second kind, wherein each vertex of said first kind represents a co-ordinate of said first polymer, and each vertex of said second kind represents a co-ordinate of said second polymer;
(ii) obtaining a plurality of edges on said bipartite graph, each connecting one vertex of first kind and one vertex of said second kind, thereby providing two connected vertices, thereby providing two connected co-ordinates.

97. The method of claim 96, wherein step (ii) comprises respectively obtaining an edge interconnecting each vertex of said first kind representing a co-ordinate in said pair of substantially congruent fragments and each vertex of said second kind representing a co-ordinate in said pair of congruent fragments, thereby respectively connecting said rigid portion of said first polymer structure and said rigid portion of said second polymer structure.

98. The method of claim 97, further comprising, for each two non-connected vertices, one vertex of said first kind and one vertex of said second kind:
(A) determining a distance between said two non-connected vertices; and
(B) if said distance is below a predetermined threshold MaxDist then establishing and edge interconnecting said two non-connected vertices, thereby providing two connected vertices.

99. The method of claim 98, further comprising finding a maximal number of vertex-disjoint edges, each vertex-disjoint edge of said plurality vertex-disjoint edges being defined such that there is no common vertex between two said vertex-disjoint edges.

100. A method of object recognition for computer vision, the object having a curve-like structure, the structure having rigid portions and flexible portions, the method being executable by a computer and comprising:
(a) obtaining a first object structure and a second object structure; and
(b) applying a semi-flexible transformation on at least a portion of said second object structure, so as to at least partially superimpose said first object and said second object, hence providing at least a partial recognition of said second object.

101. The method of claim 100, wherein said rigid portions are selected from the group consisting of predefined rigid portions and non-predefined rigid portions.

102. The method of claim 100, wherein said flexible portions are selected from the group consisting of predefined flexible portions and non-predefined flexible portions.

103. The method of claim 100, wherein said applying semi-flexible transformation comprises using a set of rigid transformations, one for each of the rigid portions.

104. Apparatus for computer recognition of objects by comparison, the object comprising a structure having rigid portions and flexible portions, the apparatus comprising:
(a) an input unit, for inputting a first object structure and a second object structure; and
(b) a transforming unit for applying a semi-flexible transformation on at least a portion of said second object structure, so as to at least partially superimpose said first object structure and said second object structure, hence to provide at least a partial recognition of said second object.

105. The apparatus of claim 104, wherein said rigid portions are selected from the group consisting of predefined rigid portions and non-predefined rigid portions.

106. The apparatus of claim 104, wherein said flexible portions are selected from the group consisting of predefined flexible portions and non-predefined flexible portions.

107. The apparatus of claim 104, wherein said transforming unit comprises a storage unit for holding a set of rigid transformations, one for each of the rigid portions.

108. An apparatus for object recognition for computer vision of objects having a curve-like structure, the apparatus comprising:
(a) an input unit for receiving sequences of co-ordinates representative of three-dimensional structure of at least a first object structure and a second object structure, each represented by a sequence of co-ordinates;
(b) a detector operable to select from each of said first and said second object structure at least one set of fragments, said detector being associated with transformation functionality to ensure that each fragment is transformable so that a fragment of said first object structure and a fragment of said second object structure are at least partially superimposed, thereby to detect at least one set of pairs of congruent fragments;
(c) an associating unit for associating at least two of said pairs of congruent fragments, to form at least one set of associated pairs of fragments; and
(d) a clustering unit for clustering each set of associated pairs of fragments to provide at least one congruent region represented by at least one associated pair of fragments;
thereby providing at least a partial recognition of said second object.

109. The apparatus of claim 108, wherein said detector comprises:
(i) a storage unit for holding a match-list comprising at least one element, each element comprising a pair of co-ordinates, one co-ordinate of said first object structure and one co-ordinate of said second object structure;
(ii) electronic-calculating functionality for determining a root-mean-square deviation of a concatenated match-list comprising said match-list and at least one additional element; and
(iii) a memory for holding instructions for setting said match-list equal to said concatenated match-list.

110. The apparatus of claim 109, wherein said instructions comprise determining whether said root-mean-square deviation is below a predefined threshold MaxRMSD, and if so then setting said match-list equal to said concatenated match-list.

111. The apparatus of claim 109, wherein said electronic-calculating functionality of said part (ii) is operable to select said at least one additional element from the group consisting of a consecutive element to the right of said match-list and a consecutive element to the left of said match-list.

112. The apparatus of claim 109, wherein said detector further comprises:
(iv) a query-length setter for setting a query-length;
(v) a memory for holding instructions for setting said match list to define a first pair of congruent fragments; and
(vi) a storage unit for holding said first pair of congruent fragments.

113. The apparatus of claim 112, wherein said instructions comprise determining whether said query-length is above a predetermined threshold MinFragSize and if so then defining said first pair of substantially congruent fragments to be equal to said match-list.

114. The apparatus of claim 112, wherein said query-length setter comprises electronic-calculating functionality for setting said query-length equal to a total number of elements of said match-list.

115. The apparatus of claim 112, wherein said storage unit is operable to hold two consecutive said pairs of substantially congruent fragments which are partially overlapped.

116. The apparatus of claim 115, wherein said overlap is smaller than a predetermined threshold MaxOverlap.

117. The apparatus of claim 115, wherein said query-length setter comprises electronic-calculating functionality for setting said query-length equal to a subtraction of half of said overlap from a length of said match-list.

118. The apparatus of claim 112, further comprising a match list initiator for setting said match list equal to a single element consecutive to a previously defined pair of congruent fragments.

119. The apparatus of claim 108, wherein said associating unit comprises:
(i) a constructor, for constructing a graph having a plurality of vertices, each vertex representing one of a respective pair of congruent fragments;
(ii) a weighter, for obtaining a plurality of directed edges on said graph each connecting two of said vertices thereby defining at each edge an incoming vertex and an outgoing vertex, and for weighting said edges using a scoring function, thereby providing a weighted acyclic directed graph;
(iii) electronic-calculating functionality for applying a single-source shortest path algorithm to said weighted acyclic directed graph thereby to provide a plurality of paths;
(iv) electronic-calculating functionality for classing said plurality of paths in accordance with a number of vertices on each of said plurality of paths, to define at least one class of paths, each class comprising at least one path;
(v) electronic-calculating functionality for determining for each path of each class of paths, a value for path weight; and
(vi) electronic-calculating functionality for sorting each class of paths using said values of path weight.

120. The apparatus of claim 119, wherein said weighter comprises:
(A) a selector, for selecting two of said plurality of vertices;
(B) electronic-calculating functionality for determining whether corresponding pairs of substantially congruent fragments are in an ascending order, said ascending order being both with respect to said co-ordinates of said first object structure, and with respect to said co-ordinates of said second object structure;
(C) an identifier, for determining a first gap between two consecutive fragments of said first object, and a second gap between two consecutive corresponding fragments of said second object; and
(D) electronic-calculating functionality for comparing said first gap with a predetermined threshold MaxGap1 and for comparing said second gap with a predetermined threshold MaxGap2.

121. The apparatus of claim 120, wherein said storage unit is operable to hold two consecutive pairs of substantially congruent fragments which are partially overlapped.

122. The apparatus of claim 121, wherein said scoring function is substantially:
−(L+1−Δ)²+max(|Gap1,|Gap2|)+∥Gap1|−|Gap2∥,
wherein:
L is a length of said pair of substantially congruent fragments, represented by said incoming vertex,
Δ is half of said overlap,
Gap1 is said first gap, and
Gap2 is said second gap.

123. The apparatus of claim 119, wherein:
said constructor is operable to construct an additional virtual vertex; and
said weighter is operable to obtain a virtual edge connecting said virtual vertex with all said plurality of vertices and to weight each said virtual edge using a virtual scoring function.

124. The apparatus of claim 123, wherein said virtual scoring function substantially equals zero.

125. The apparatus of claim 108, wherein said clustering unit comprises:
(i) a storage unit for storing a query-region comprising at least one associated pair of fragments;
(ii) a transforming unit for simultaneously transforming said query-region using a rigid transformation so as to obtain a superimposition of all of said associated pairs of fragments within said query-region;
(iii) electronic-calculating functionality for determining a query-region root-mean-square deviation;
(iv) a memory for holding instructions for setting one congruent region equal to said query-region; and
(v) a storage unit for storing each congruent region.

126. The apparatus of claim 125, wherein said instructions of part (iv) comprise: determining whether said query-region root-mean-square deviation is below a predetermined threshold MaxRMSD, and if so then setting one congruent region equal to said query-region.

127. The apparatus of claim 125, wherein said clustering unit further comprising a query-region initiator for setting said query-region equal to a first associated pair of fragments.

128. The apparatus of claim 125, wherein said clustering unit further comprising a query-region initiator for setting said query-region equal to an associated pair of fragments consecutive to an existing one of said congruent regions.

129. A method of object recognition for computer vision, the object having a curve-like structure, the method being executable by a computer and comprising:
(a) receiving a first object structure and a second object structure, each represented by a sequence of co-ordinates;
(b) for each said object structure, detecting at least one set of fragments, wherein each fragment of a respective sequence is respectively transformable so that a fragment of said first object structure and a fragment of said second object structure are at least partially superimposed, thereby providing at least one set of pairs of substantially congruent fragments;
(c) for each set of pairs of substantially congruent fragments, mutually associating at least two pairs of said set of pairs, thereby providing at least one set of associated pairs of fragments; and
(d) for each set of pairs of substantially congruent fragments clustering each of said set of associated pairs of fragments, thereby providing at least one congruent region represented by at least one associated pair of fragments;
hence providing at least a partial recognition of said second object.

130. The method of claim 129, wherein said at least partially superimposed comprises a small overall root-mean-square deviation.

131. The method of claim 129, wherein step (b) comprises:
(i) obtaining a match-list comprising at least one element, each element comprising a pair of co-ordinates, one co-ordinate of said first object structure and one co-ordinate of said second object structure;
(ii) determining a root-mean-square deviation of a concatenated match-list comprising said match-list and at least one additional element; and
(iii) if said root-mean-square deviation is below a predefined threshold MaxRMSD, then setting said match-list equal to said concatenated match-list.

132. The method of claim 131, wherein steps (i)-(iii) are sequentially repeated at least once.

133. The method of claim 131, wherein said at least one additional element is selected from the group consisting of a consecutive element to the right of said match-list and a consecutive element to the left of said match-list.

134. The method of claim 132, further comprising determining a query-length wherein if said query-length is above a predetermined threshold MinFragSize then defining said pair of substantially congruent fragments to be equal to said match-list.

135. The method of claim 132, wherein said query-length substantially equals a length of said match-list.

136. The method of claim 134, wherein two consecutive said pairs of substantially congruent fragments are partially overlapped.

137. The method of claim 136, wherein said overlap is smaller than a predetermined threshold MaxOverlap.

138. The method of claim 136, wherein said query-length equals the subtraction of half of said overlap from a length of said match-list.

139. The method of claim 134, wherein said match-list is initiated by a seed comprising a first seed co-ordinate and a second seed co-ordinate.

140. The method of claim 139, wherein said first and said second seed co-ordinates are respectively consecutive to a previously defined pair of congruent fragments.

141. The method of claim 139, wherein said first seed co-ordinate is a first co-ordinate of said first object.

142. The method of claim 139, wherein said second seed co-ordinate is a first co-ordinate of said second object.

143. The method of claim 129, wherein said associating comprises:
(i) constructing a graph having a plurality of vertices, each vertex representing one of said pair of congruent fragments;
(ii) obtaining a plurality of directed edges on said graph each connecting two of said vertices and defining an incoming vertex and an outgoing vertex, wherein each said edge is weighted using a scoring function, thereby providing a weighted acyclic directed graph;
(iii) applying a single-source shortest path algorithm to said weighted acyclic directed graph thereby providing a plurality of paths;
(iv) classing said plurality of paths decreasingly in accordance with a number of vertices on each of said plurality of paths, thereby defining at least one class of paths, each class comprising at least one path;
(v) for each class of paths, determining for said path, a value for path weight; and
(vi) for each class of paths, sorting each path using said values of path weight;
thereby providing at least one set of associated pairs of fragments.

144. The method of claim 143, wherein said obtaining a plurality of directed edges on said graph comprises:
(A) selecting two of said vertices;
(B) determining whether corresponding pairs of substantially congruent fragments are in an ascending order, said ascending order being both with respect to said co-ordinates of said first object structure, and with respect to said co-ordinates of said second object structure;
(C) determining a first gap and a second gap; and
(D) then, if said corresponding pairs of substantially congruent fragments are in said ascending order and if said first gap is smaller than a predetermined threshold MaxGap1 and if said second gap is smaller than a predetermined threshold MaxGap2, then obtaining a directed edge between said two vertices.

145. The method of claim 144, wherein each of said first and said second gap are respectively structurally dissimilar fragments of said first and said second object structures, said structurally dissimilar fragments being between said corresponding pairs of substantially congruent fragments which are in said ascending order.

146. The method of claim 144, wherein two consecutive pairs of substantially congruent fragments are partially overlapped.

147. The method of claim 146, wherein said scoring function is substantially:
−(L+1−Δ)²+max(|Gap1|,|Gap2|)+∥Gap1|−|Gap2∥,
wherein L is a length of said pair of substantially represented by said incoming vertex, where Δ is half of said overlap, where Gap1 is said first gap and where Gap2 is said second gap.

148. The method of claim 143, further comprising adding a virtual vertex to said weighted acyclic directed graph, said virtual vertex being connected by a virtual edge to all of said vertices, wherein said virtual edge is weighted by a virtual scoring function.

149. The method of claim 148, wherein said virtual scoring function substantially equals zero.

150. The method of claim 129, wherein said clustering comprises:
(i) establishing a query-region comprising a seed of an associated pair of fragments;
(ii) concatenating an additional associated pair of fragments to said query-region;
(iii) simultaneously transforming said query-region using a rigid transformation so as to obtain a superimposition of all of said associated pairs of fragments within said query-region;
(iv) determining a region root-mean-square deviation; and
(v) if said region root-mean-square deviation is below a predetermined threshold MaxRMSD, then setting one congruent region equal to said query-region.

151. The method of claim 150, wherein steps (ii) to (v) are repeated at least once.

152. The method of claim 150, wherein said seed of associated pair of fragments of step (i) comprises a first associated pair of fragments.

153. The method of claim 150, wherein said seed of associated pair of fragments of step (i) comprises an associated pair of fragments consecutive to an existing one of said congruent regions.

154. Apparatus for object recognition for computer vision of objects having a curve-like structure, the apparatus comprising:
(a) an input unit for receiving sequences of co-ordinates representative of three-dimensional structure of at least a first object structure and a second object structure, each represented by a sequence of co-ordinates;
(b) a detector operable to select from each of said first and said second object structure at least one set of fragments, said detector being associated with transformation functionality to ensure that each fragment is transformable, using a rigid transformation, so that a fragment of said first object structure and a fragment of said second object structure are at least partially superimposed, thereby to detect at least one set of rigid transformations;
(c) a transforming unit for applying at least one of said set of rigid transformations over a plurality of fragments of said first object structure, a plurality of fragments of said second object structure or a plurality of fragments of both said first and said second object structures, so as to at least partially superimpose said first object structure and said second object structure, thereby to provide at least a partial alignment of said first and said second object structures.

155. The apparatus of claim 154, wherein said detector comprises:
(i) a storage unit for holding a match-list comprising at least one element, each element comprising a pair of co-ordinates, one co-ordinate of said first object structure and one co-ordinate of said second object structure;
(ii) electronic-calculating functionality for determining a root-mean-square deviation of a concatenated match-list comprising said match-list and at least one additional element; and
(iii) a memory for holding instructions for setting said match-list equal to said concatenated match-list.

156. The apparatus of claim 155, wherein said instructions comprise determining whether said root-mean-square deviation is below a predefined threshold MaxRMSD, and if so then setting said match-list equal to said concatenated match-list.

157. The apparatus of claim 155, wherein said electronic-calculating functionality of said part (ii) is operable to select said at least one additional element from the group consisting of a consecutive element to the right of said match-list and a consecutive element to the left of said match-list.

158. The apparatus of claim 155, wherein said detector further comprises:
(iv) a query-length setter for setting a query-length;
(v) a memory for holding instructions for setting said match list to define a first pair of congruent fragments; and
(vi) a storage unit for holding said first pair of congruent fragments.

159. The apparatus of claim 158, wherein said instructions comprise determining whether said query-length is above a predetermined threshold MinFragSize and if so then defining said first pair of substantially congruent fragments to be equal to said match-list.

160. The apparatus of claim 158, wherein said query-length setter comprises electronic-calculating functionality for setting said query-length equal to a total number of elements of said match-list.

161. The apparatus of claim 158, further comprising a match list initiator for setting said match list equal to a single element consecutive to a previously defined pair of congruent fragments.

162. The apparatus of claim 154, wherein said transforming unit comprises:
(i) a constructor, for constructing a bipartite graph having a plurality of vertices of a first kind and a plurality of vertices of a second kind, said constructor being operable to ensure that each vertex of said first kind represents one co-ordinate of said first object, and each vertex of said second kind represents one co-ordinate of said second object;
(ii) a weighter, for obtaining a plurality of edges on said bipartite graph, each connecting one vertex of said first kind and one vertex of said second kind, thereby providing two connected vertices, thereby providing two connected co-ordinates.

163. The apparatus of claim 162, wherein said weighter comprises:
(A) a selector for selecting two non-connected vertices, one vertex of said first kind and one vertex of said second kind;
(B) electronic-calculating functionality for determining a distance between said two non-connected vertices;
(C) a memory for storing instructions for establishing an edge interconnecting said two non-connected vertices; and
(D) a storage unit for holding said edge.

164. The apparatus of claim 163, wherein said instructions of part (C) comprise determining whether said distance is below a predetermined threshold MaxDist, and if so then establishing said edge interconnecting said two non-connected vertices, thereby providing two connected vertices.

165. The apparatus of claim 163, wherein said transforming unit further comprises electronic calculating functionality for finding a maximal number of vertex-disjoint edges, each vertex-disjoint edge of said plurality vertex-disjoint edges being defined such that there is no common vertex between two said vertex-disjoint edges.

166. A method of object recognition for computer vision, the object having a curve-like structure, the method being executable by a computer and comprising:
(a) receiving a first object structure and a second object structure, each represented by a sequence of co-ordinates;
(b) for each said object structure, detecting at least one set of fragments, wherein each fragment of a respective sequence is respectively transformable using a rigid transformation, so that a fragment of said first object structure and a fragment of said second object structure are at least partially superimposed, thereby providing at least one set of pairs of substantially congruent fragments, each characterized by a rigid transformation, hence providing at least one rigid transformation; and
(c) applying at least one of said at least one rigid transformation over a plurality of fragments of said first object structure, a plurality of fragments of said second object structure or a plurality of fragments of both said first and said second object structures, so as to at least partially superimpose said first object structure and said second object structure, thereby to provide at least a partial alignment of said first and said second object structures.

167. The method of claim 166, wherein steps (b) and (c) are repeated at least once.

168. The method of claim 166, wherein said at least partially superimposed sequence comprises a small overall root-mean-square deviation.

169. The method of claim 166, wherein step (b) comprises,
(i) obtaining a match-list comprising at least one element, each element comprising a pair of co-ordinates, one co-ordinate of said first object structure and one co-ordinate of said second object structure;
(ii) determining a root-mean-square deviation of a concatenated match-list comprising said match-list and at least one additional element; and
(iii) if said root-mean-square deviation is below a predefined threshold MaxRMSD, then setting said match-list equal to said concatenated match-list.

170. The method of claim 169, wherein steps (ii) and (iii) are sequentially repeated at least once.

171. The method of claim 169, wherein said at least one additional element is selected from the group consisting of a consecutive element to the right of said match-list and a consecutive element to the left of said match-list.

172. The method of claim 170, further comprising determining a query-length wherein if said query-length is above a predetermined threshold MinFragSize then setting said pair of substantially congruent fragments equal to said match-list.

173. The method of claim 172, wherein said query-length substantially equals a length of said match-list.

174. The method of claim 169, wherein said match-list comprises a single element consecutive to an existing one of said pairs of congruent fragments.

175. The method of claim 166, wherein step (c) comprises:
(i) obtaining a bipartite graph having a plurality of vertices of a first kind and a plurality of vertices of a second kind, wherein each vertex of said first kind represents a co-ordinate of said first object, and each vertex of said second kind represents a co-ordinate of said second object;
(ii) obtaining a plurality of edges on said bipartite graph, each connecting one vertex of said first kind and one vertex of said second kind, thereby providing two connected vertices, thereby providing two connected co-ordinates.

176. The method of claim 175, wherein step (ii) comprises respectively obtaining an edge interconnecting each vertex of said first kind representing a co-ordinate in said pair of substantially congruent fragments and each vertex of said second kind representing a co-ordinate in said pair of congruent fragments, thereby respectively connecting said rigid portion of said first object structure and said rigid portion of said second object structure.

177. The method of claim 176, further comprising, for each two non-connected vertices, one of said vertices of said first kind and one of said vertices of said second kind:
(A) determining a distance between said two non-connected vertices; and
(B) if said distance is below a predetermined threshold MaxDist then establishing and edge interconnecting said two non-connected vertices, thereby providing two connected vertices.

178. The method of claim 176, further comprising finding a maximal number of vertex-disjoint edges, each vertex-disjoint edge of said plurality vertex-disjoint edges being defined such that there is no common vertex between two said vertex-disjoint edges.
